(19) United States
(12) Patent Application Publication
(10) Pub. No.: US 2004/0162263 A1
(43) Pub. Date: Aug. 19, 2004

(54) Pharmaceutical formulations targeting specific regions of the gastrointesinal tract

(75) Inventors: Howard Sands, Wilmington, DE (US); Sanjeev Redkar, Union City, CA (US); Harish Ravivarapu, Union City, CA (US)

Correspondence Address: WILSON SONSINI GOODRICH & ROSATI, 650 PAGE MILL ROAD, PALO ALTO, CA, 943041050

(73) Assignee: SuperGen, Inc., a Delaware Corporation, Dublin, CA

(21) Appl. No.: 10/698,983

(22) Filed: Oct. 31, 2003

Related U.S. Application Data
(60) Provisional application No. 60/423,179, filed on Oct. 31, 2002.

(51) Int. Cl. A61K 31/7076; A61K 31/7072; A61K 9/24

(52) U.S. Cl. 514/46; 514/50; 424/471

(57) ABSTRACT

Oral formulations of pharmaceuticals are provided with enhanced bioavailability by targeting specific regions of the gastrointestinal tract. Particularly, water soluble and acid-labile drugs such as cytidine analogs (e.g., decitabine) and 2′-deoxyadenosine analogs (e.g., pentostatin) are formulated with pH-sensitive polymers so that these drugs are preferably absorbed in the upper regions of the small intestine, such as the jejunum. In addition, drugs with poor oral bioavailability such as camptothecin compounds (e.g., 9-nitro-camptothecin) can also be formulated using similar strategies in order to significantly improve their oral bioavailability. These formulations can be used to treat a wide variety of diseases or conditions, such hematological disorders, benign tumors, cancer, restenosis, inflammatory diseases, and autoimmune diseases.

### CROSS-REFERENCE TO RELATED APPLICATION

This application claims priority to U.S. Provisional Application No. 60/423,179, filed Oct. 31, 2002, which is hereby incorporated by reference herein.

### BACKGROUND OF THE INVENTION

1. Field of the Invention

This invention relates to pharmaceutical formulations for oral delivery of drugs to specific regions of the gastrointestinal tract for enhanced bioavailability, and more particularly relates to oral formulations of water-soluble, acid-labile drugs such as cytidine analogs (e.g., decitabine) and 2′-deoxyadenosine analogs (e.g., pentostatin), as well as drugs with poor bioavailability such as camptothecin compounds.

2. Description of the Related Art

1. Decitabine

Decitabine, 5-aza-2′-deoxycytidine, is an antagonist of its related natural nucleoside, deoxycytidine. The only structural difference between these two compounds is the presence of a nitrogen at position 5 of the cytosine ring in decitabine as compared to a carbon at this position for deoxycytidine. Two isomeric forms of decitabine can be distinguished. The β-anomer is the active form. The modes of decomposition of decitabine in aqueous solution are (a) conversion of the active β-anomer to the inactive α-anomer (Pompon et al. (1987) J. Chromat. 388:113-122); (b) ring cleavage of the aza-pyrimidine ring to form N-(formylamidino)-N′-β-D-2′-deoxy-(ribofuranosy)-urea (Mojaverian and Repta (1984) J. Pharm. Pharmacol. 36:728-733); and (c) subsequent forming of guanidine compounds (Kissinger and Stemm (1986) J. Chromat. 353:309-318).

Decitabine possesses multiple pharmacological characteristics. At a molecular level, it is S-phase dependent for incorporation into DNA. At a cellular level, decitabine can induce cell differentiation and exert hematological toxicity. Despite having a short half life in vivo, decitabine has an excellent tissue distribution.

The most prominent function of decitabine is its ability to specifically and potently inhibit DNA methylation. Methylation of cytosine to 5-methylcytosine occurs at the level of DNA. Inside the cell, decitabine is first converted into its active form, the phosphorylated 5-aza-deoxycytidine, by deoxycytidine kinase which is primarily synthesized during the S phase of the cell cycle. The affinity of decitabine for the catalytical site of deoxycytidine kinase is similar to the natural substrate, deoxycytidine. Momparler et al. (1985) 30:287-299. After conversion to its triphosphate form by deoxycytidine kinase, decitabine is incorporated into replicating DNA at a rate similar to that of the natural substrate, dCTP. Bouchard and Momparler (1983) Mol. Pharmacol. 24:109-114.

Incorporation of decitabine into the DNA strand has a hypomethylation effect. Each class of differentiated cells has its own distinct methylation pattern. After chromosomal duplication, in order to conserve this pattern of methylation, the 5-methylcytosine on the parental strand serves to direct methylation on the complementary daughter DNA strand. Substituting the carbon at the 5 position of the cytosine for a nitrogen interferes with this normal process of DNA methylation. The replacement of 5-methylcytosine with decitabine at a specific site of methylation produces an irreversible inactivation of DNA methyltransferase, presumably due to formation of a covalent bond between the enzyme and decitabine. Juttermann et al. (1994) Proc. Natl. Acad. Sci. USA 91:11797-11801. By specifically inhibiting DNA methyltransferase, the enzyme required for methylation, the aberrant methylation of the tumor suppressor genes can be prevented.

Decitabine is commonly supplied as a sterile lyophilized powder for injection, together with buffering salt, such as potassium dihydrogen phosphate, and pH modifier, such as sodium hydroxide. For example, decitabine is supplied by SuperGen, Inc., as lyophilized powder packed in 20 mL glass vials, containing 50 mg of decitabine, monobasic potassium dihydrogen phosphate, and sodium hydroxide. When reconstituted with 10 mL of sterile water for injection, each mL contain 5 mg of decitabine, 6.8 mg of KH₂PO₄, and approximately 1.1 mg NaOH. The pH of the resulting solution is 6.5-7.5. The reconstituted solution can be further diluted to a concentration of 1.0 or 0.1 mg/mL in cold infusion fluids, i.e., 0.9% Sodium Chloride; or 5% Dextrose; or 5% Glucose; or Lactated Ringer's. The unopened vials are typically stored under refrigeration (2-8° C.; 36-46° F.), in the original package.

Decitabine is most typically administrated to patients by injection, such as by a bolus I.V. injection, continuous I.V. infusion, or I.V. infusion. The length of I.V. infusion is limited by decitabine's decomposition in aqueous solutions.

It has been found that when 5-azacytidine (azaC) is orally administered (8 mg/kg) to repeatedly phlebotomized baboon (PCV less than 20%) there is no elevation in the fetal hemoglobin levels (Hb F), indicating very minimal oral bioavailability. DeSimone et al (1985) American. J. of Hem. 18:283-288. AzaC is more active when administered parenterally than orally in the treatment of L1210 leukemic mice due to poor bioavailability. Neil at al (1975) Cancer Chemother. Rep. 59:459-465. In L1210 leukemic mice, peroral doses of cytarabine (cytosine arbinaoside) required to elicit an anti-tumor effect are about 3 to 10 times those required when administered parenterally. Neil et al. (1970) Cancer Research 30:2166-2172. The poor bioavailability of such cytidine analogs is presumably due to the degradation of the cytidine analog by cytidine deaminases as well as their inherent chemical instability in the acidic gastric environment.

2. 2′-Deoxyadenosine Analogs

Certain 2′-deoxyadenosine analogs have been found to have very useful clinical pharmacological benefits. These include, but are not limited to, 2′-deoxycoformycin (also referred to as dCF, pentostatin, or NIPENT®), an inhibitor of adenosine deaminase; fludarabine monophosphate (FLU), a fluorinated analogue of adenine that is relatively resistant to adenosine-deaminase and 2-chloro-2′-deoxyadenosine (also known as cladribine or 2CDA) a drug also resistant to adenosine deaminase through introduction of a chlorine at the 2 carbon.

In humans, these compounds are assumed to act through a number of adenosine related pathways, particularly the adenosine deaminase (ADA) pathway. A genetic deficiency of ADA may cause severe combined immunodeficiency. Dighiero, G., “Adverse and beneficial immunological effects of purine nucleoside analogues,” Hematol Cell Ther, 38:575-581 (1996).

While the exact nature of the ADA pathway intervention seems unclear, it may be that analogs of adenosine resistant to cellular deamination might mimic the ADA-deficient state. Lack of ADA seems to lead to a build up of deoxyadenosine and adenosine triphosphate in the cell, thus fatally accelerating DNA strand breaks in the cell. Under normal conditions, cells are continuously breaking and rejoining DNA. When this physiological process is accelerated by the effect of excess adenosine triphosphate, it leads to consumption of NAD for poly-ADP-ribose synthesis. This polymer is produced from nicotinamide adenosine dinucleotides (NAD) in a reaction catalyzed by the chromatin-associated poly(ADP-ribose) synthetase, leading to a depletion of the NAD content of the cell. This depletion induces a profound alteration of cellular reducing power, because of lethal ADP and ATP depletion.

The result is programmed cell death through activation of a Ca²⁺, Mg²⁺-dependent endonuclease. Hence, it appears that nucleoside analogs according to the invention can act on cells, with preferential lymphocytic activity, via an apoptotic process. The fact that supplementation of a cell medium with the NAD precursor of nicotinamide or 3-aminobenzamide, an inhibitor of poly (ADP-ribose) synthetase, prevented NAD depletion and reduces 2CDA toxicity, tends to support this hypothesis.

The various 2′-deoxyadenosine analogs affect the ADA pathway in different manners. DCF, for example, has been shown to be an quasi-irreversible inhibitor of ADA. By favoring the predominance of deoxycytidine kinase (DCK) over the dephosphorylating enzyme 5-nucleotidase in lymphocytes it induces a preferential accumulation of deoxyadenosine-5′-triphosphate (dATP). By comparison, FLU and 2CDA are rather resistant to the enzyme. Both drugs are initially phosphorylated by DCK and contribute to the accumulation of cellular adenosine triphosphate surrogates. As noted above, the accumulation of adenosine triphosphate, whether by the presumed DCF mechanism, or the FLU or 2CDA mechanism, promotes the apoptotic death of the cell.

A problem with administering these 2-deoxyadenosine analogs is their dosage form. Currently, these analogs are available only in an intravenous (IV) dosage form. While this dosage form is customary, especially for use in oncology indications, it is limiting in a variety of ways. For example, IV dosing is expensive. It requires a highly trained medical professional to administer the IV dose. The dosing involves expensive equipment and materials. Additionally, IV dosing presents increased possibilities of infection, through use of contaminated equipment or accidental contamination, for example. This is a special concern in health care settings where increased incidences of antibiotic resistant bacteria are being noted.

A seemingly natural solution to the IV dosage problem is the development of an oral dosage form. Such a dosage form alleviates most, if not all, of the above-mentioned problems associated with IV or other parenteral dosage forms. However, the art recognized serious problems with the development of an oral dosage form. Chief among these is that adenosine analogs have been known for years to be susceptible to acid-catalyzed glycosidic cleavage. Therefore, one of skill in the art would expect that an orally administered adenosine analog would be cleaved in the stomach, and rendered inactive.

For example, investigators studying 2′-deoxycoformycin have not considered oral administration of the drug worth studying because of its known acid lability. Marvin M. Chassin et al. Biochemical Pharmacology 28:1849-1855 (1979). Likewise, other researchers have reported on the acid lability of 2′-deoxycoformycin. L. A. al-Razzak et al. 7:452-460 (1990).

Other adenosine analogs may be expected to have similar acid lability characteristics. A. Tarasiuk et al. Arch. Immunol. Ther. Exp. (Warsz) 42:13-15 (1994); T. Ono Nucleic Acids Res. 25:4581-4588 (1997).

3. Camptothecin Compounds

The original Camptothecin was isolated from the plant, Camptotheca acuminata, in the 1960's (Wall, M. et al. (1966) J. Am. Chem. Soc. 88: 3888-3890). Camptothecin has a pentacyclic ring system with only one asymmetric center in ring E with a 20(S)-configuration. The pentacyclic ring system includes a pyrrole quinoline moiety (rings A, B and C), a conjugated pyridone (ring D), and a six-membered lactone (ring E) with an α-hydoxyl group.

Camptothecin itself is highly lipophilic and poorly water-soluble. Sodium camptothecin that is solubilized by sodium hydroxide in water was used in clinical trials in the early 70's and found to have antitumor activity. However, this formulation of camptothecin administered via i.v. caused unpredictable side effects such as myelosuppression and hemorrhagic cystitis. Clinical trials with sodium camptothecin were eventually discontinued because of these toxicities and the lack of consistent antitumor activity.

Continued evaluation of this agent showed that the sodium carboxylate salt is only 10% as potent as the native camptothecin with the closed lactone ring intact (Wall et al. in (1969) “International Symposium on Biochemistry and Physiology of the Alkaloids, Mothes et al. eds. Academic Verlag, Berlin, 77; Giovanella et al. (1991) Cancer Res. 51:3052). Studies also showed that camptothecin and its derivatives undergo an alkaline hydrolysis of the E-ring lactone, resulting in a carboxylate form of camptothecin. At pH levels below 7.0, the lactone E-ring form of camptothecin predominates. However, intact lactone ring E and α-hydoxyl group have been shown to be essential for antitumor activity of camptothecin and its derivatives.

Camptothecin and its derivatives have been shown to inhibit DNA topoisomerase I by stabilizing the covalent complex (“cleavable complex”) of enzyme and strand-cleaved DNA. Inhibition of topoisomerase I by camptothecin induces protein-associated DNA single-stran breaks which occur during the S-phase of the cell cycle. Since the S-phase is relatively short compared to other phases of the cell cycle, longer exposure to camptothecin should result in increased cytotoxicity of tumor cells. Studies indicate that only the closed lactone form of the drug helps stabilize the cleavable complex, leading to inhibition of the cell cycle and apoptosis.

To preserve the lactone form of camptothecin, camptothecin and its water insoluble derivatives have been dissolved in N-methyl-2-pyrrolidinone in the presence of an acid (U.S. Pat. No. 5,859,023). Upon dilution with an acceptable parenteral vehicle, a stable solution of camptothecin was obtained. The concentrated solution of camptothecin was also filled in gel capsules for oral administration. It is believed that such formulations increase the amount of lipophilic lactone form of camptothecin that diffuse through the cellular and nuclear membranes in tumor cells.

### SUMMARY OF THE INVENTION

The present invention provides innovative oral formulations of pharmaceuticals with enhanced bioavailability by targeting specific regions of the gastrointestinal tract. Particularly, water soluble and acid-labile drugs such as cytidine analogs (e.g., decitabine and 5′-azacytidine) and 2′-deoxyadenosine analogs (e.g., pentostatin) are formulated with pH-sensitive polymers so that these drugs are preferably absorbed in the upper regions of the small intestine, such as the jejunum. In addition, drugs with poor oral bioavailability such as camptothecin compounds (e.g., 9-nitro-camptothecin) can also be formulated using similar strategies in order to significantly improve their oral bioavailability.

In one aspect of the invention, a pharmaceutical composition is provided. The pharmaceutical composition comprises: a water-soluble, acid-labile drug enteric-coated with a coating material that dissolves at pH above about 5.2.

According to the invention, the solubility of the drug is preferably above 1 mg/ml in water or aqueous solution, more preferably above 5 mg/ml in water or aqueous solution, and most preferably above 10 mg/ml in water or aqueous solution.

Also according to the invention, the drug is labile preferably at pH lower than 5.0, more preferably at pH lower than 4.0, and most preferably at pH lower than 2.0.

Examples of the drug includes, but are not limited to, cytidine analogs or derivatives such as 5-azacytidine and 5-aza-2′-deoxycytidine (or decitabine), and 2′-deoxyadenosine analogs and derivatives such as 2′-deoxycoformycin (or pentostatin), fludarabine monophosphate, and 2-chloro-2′-deoxyadenosine (or cladribine).

The coating material for enteric-coating of the drug is pH-sensitive and preferably or selectively dissolves at a threshold pH above about 5.2, optionally at pH above about 5.5, optionally at pH above about 5.8, optionally at pH above about 6.0, optionally at pH above about 6.2, optionally at pH above about 6.5, optionally at pH above about 6.5, and most preferably at pH above about 6.8, or optionally at pH above about 7.0. The pharmaceutical composition is preferred to substantially disintegrate in an aqueous medium at a pH equal or above the threshold pH within 3 hours, optionally within 2 hours, optionally within 1 hour, more preferably within 30 min, and most preferably within 15 mm.

Examples of such a coating material include, but are not limited to, cellulose phthalates that selectively dissolve at pH above 5.6, the Eudragit® family of polymers (e.g., Eudragit L30D with threshold pH of 5.6, Eudragit L with threshold pH of 6.0, and Eudragit S with threshold pH of 6.8), Aquateric with threshold pH of 5.8, polyvinylacetate phthalate (PVAP) that releases drug at pH values above about 5.0, Shellac® that releases the drug at about pH7.0, and cellulose acetate phthalate (CAP) with threshold pH of 6.0.

In a preferred embodiment, the drug is enteric-coated with Eudragit L100 with the threshold pH of 6.0 or L100-55 with a threshold pH of 5.5.

Also according to the invention, the pharmaceutical composition is preferred not to substantially disintegrate in an acidic, aqueous medium at pH 1.0-3.0 for at least 1 hour, more preferred not to substantially disintegrate in an acidic, aqueous medium at pH 1.2-2.0 for at least 1 hour, more preferably for at least 2 hours, and most preferably for at least 3 hours. Optionally, the pharmaceutical formulation does not substantially disintegrate in an acidic, aqueous medium at pH 1.2-1.5 for at least 1 hour, more preferably for at least 2 hours, and most preferably for at least 3 hours. The composition is considered to be substantially disintegrated if at least 50% of the composition disintegrates, e.g., undergoes rupture.

In addition, the pharmaceutical composition preferably disintegrates substantially in an aqueous medium at pH 5.2-7.5 within 1 hour, more preferably disintegrates substantially in an aqueous medium at pH 6.0-7.2 within 30 minutes, and most preferably disintegrates substantially in an aqueous medium at pH 6.5-7.0 within 15 minutes.

The amount of the enteric-coating material is preferably 1-10% w/w in the composition, more preferably 2-8% w/w in the composition, and most preferably 3-6% w/w in the composition.

The pharmaceutical composition may be in a form of tablet or capsule. In a preferred embodiment, the composition is in a form of tablet. The hardness of the tablet without the enteric-coat is preferably at least 4 kp, more preferably at least 8 kp, and most preferably 10 kp. The size of the tablet is preferably 5-20 mm, more preferably 8-15 mm, and most preferably 10-13 mm.

In any of the above dosage forms, the concentration of the drug is preferably 0.1-20% w/w, optionally 1-10% w/w, or optionally 2-5% w/w.

Optionally, the pharmaceutical composition may further comprise a seal-coating material that seals the drug to prevent decomposition due to exposure to moisture, such as hydroxy propylmethylcellulose. Optionally, the pharmaceutical composition may further comprise buffer salt such as potassium or sodium phosphate in an amount sufficient to maintain the pH of the local environment to be 5.2-7.0 when the pharmaceutical composition is dissolved in the GI tract. Examples of such buffer salts include, but are not limited to, KH₂PO₄ and Na₂HPO₄.

In another aspect of the invention, a pharmaceutical composition for delivering a camptothecin compound in vivo is provided. The pharmaceutical composition comprises: a camptothecin compound enteric-coated with an enteric coating material that dissolves at pH above 5.2.

The enteric coating material for enteric-coating of the camptothecin compound is pH-sensitive and preferably or selectively dissolves at pH above about 5.2, preferably at pH above about 5.8, more preferably at pH above about 6.0, and most preferably at pH above about 6.4.

The enteric coating material for enteric-coating of the drug is pH-sensitive and preferably or selectively dissolves at a threshold pH above about 5.2, optionally at pH above about 5.5, optionally at pH above about 5.8, optionally at pH above about 6.0, optionally at pH above about 6.2, optionally at pH above about 6.5, optionally at pH above about 6.5, and most preferably at pH above about 6.8, optionally at pH above about 7.0, optionally at pH above about 7.2, or optionally at pH above about 7.5. The pharmaceutical composition is preferred to substantially disintegrate in an aqueous medium at a pH equal or above the threshold pH within 3 hours, optionally within 2 hours, optionally within 1 hour, more preferably within 30 min, and most preferably within 15 min.

Examples of such a coating material include, but are not limited to, cellulose phthalates that selectively dissolve at pH above 5.6, the Eudragit® family of polymers (e.g., Eudragit L30D with threshold pH of 5.6, Eudragit L with threshold pH of 6.0, and Eudragit S with threshold pH of 6.8), Aquateric with threshold pH of 5.8, polyvinylacetate phthalate (PVAP) that releases drug at pH values above about 5.0, Shellac® that releases the drug at about pH7.0, and cellulose acetate phthalate (CAP) with threshold pH of 6.0.

In a preferred embodiment, the drug is enteric-coated with Eudragit L100 with the threshold pH of 6.0 or Eudragit L100-55 with a threshold pH of 5.5.

Also according to the invention, the pharmaceutical composition is preferred not to substantially disintegrate in an acidic, aqueous medium at pH 1.0-3.0 for at least 1 hour, more preferred not to substantially disintegrate in an acidic, aqueous medium at pH 1.2-2.0 for at least 1 hour, more preferably for at least 2 hours, and most preferably for at least 3 hours. Optionally, the pharmaceutical formulation does not substantially disintegrate in an acidic, aqueous medium at pH 1.2-1.5 for at least 1 hour, more preferably for at least 2 hours, and most preferably for at least 3 hours. The composition is considered to be substantially disintegrated if at least 50% of the composition disintegrates, e.g., undergoes rupture.

In addition, the pharmaceutical composition preferably disintegrates substantially in an aqueous medium at pH 5.2-7.5 within 1 hour, more preferably disintegrates substantially in an aqueous medium at pH 6.0-7.2 within 30 minutes, and most preferably disintegrates substantially in an aqueous medium at pH 6.5-7.0 within 15 minutes.

The amount of the enteric-coating material is preferably 1-10% w/w in the composition, more preferably 2-8% w/w in the composition, and most preferably 3-6% w/w in the composition.

The camptothecin compound may be the original 20(S)-camptothecin isolated from the plant, Camptotheca acuminata, analogs of 20(S)-camptothecin, derivatives of 20(S)-camptothecin, prodrugs of 20(S)-camptothecin, and pharmaceutically active metabolites of 20(S)-camptothecin.

Examples of camptothecin derivatives include, but are not limited to, 9-nitro-20(S)-camptothecin, 9-amino-20(S)-camptothecin, 9-methyl-camptothecin, 9-chloro-camptothecin, 9-flouro-camptothecin, 7-ethyl camptothecin, 10-methyl-camptothecin, 10-chloro-camptothecin, 10-bromo-camptothecin, 10-fluoro-camptothecin, 9-methoxy-camptothecin, 11-fluoro-camptothecin, 7-ethyl-10-hydroxy camptothecin, 10,11-methylenedioxy camptothecin, and 10,11-ethylenedioxy camptothecin, and 7-(4-methylpiperazinomethylene)-10,11-methylenedioxy camptothecin. Prodrugs of camptothecin include, but are not limited to, esterified camptothecin derivatives as decribed in U.S. Pat. No.5,731,316, such as camptothecin 20-O-propionate, camptothecin 20-O-butyrate, camptothecin 20-O-valerate, camptothecin 20-O-heptanoate, camptothecin 20-O-nonanoate, camptothecin 20-O-crotonate, camptothecin 20-O-2′,3′-epoxy-butyrate, nitrocamptothecin 20-O-acetate, nitrocamptothecin 20-O-propionate, and nitrocamptothecin 20-O-butyrate.

In particular, when substituted camptothecins are used, a large range of substitutions may be made to the camptothecin scaffold, while still retaining activity. In a preferable embodiment, the camptothecin scaffold is substituted at the 7, 9, 10, 11, and/or 12 positions. Such preferable substitutions may serve to provide differential activities over the unsubstituted camptothecin compound. Especially preferable are 9-nitrocamptothecin, 9-aminocamptothecin, 10,11-methylendioxy-20(S)-camptothecin, topotecan, irinotecan, 7-ethyl-10-hydroxy camptothecin, or another substituted camptothecin that is substituted at least one of the 7, 9, 10, 11, or 12 positions.

According to the invention, the camptothecin compound is preferably a water-insoluble camptothecin compound such as 9-nitrocamptothecin and 9-aminocamptothecin.

In any of the above dosage forms, the concentration of the camptothecin compound is preferably 0.01-20% w/w, optionally 0.1-10% w/w, or optionally 0.2-5% w/w.

According to any of the above embodiments of the invention, the pharmaceutical composition may further comprise one or more pharmaceutically acceptable excipient. The excipient may be a diluent, lubricant, disintegrant, glidant, and/or a retention-enhancing excipient.

Examples of the diluent include, but are not limited to, microcrystalline cellulose, lactose monohydrate, starch, gelatin, gum, tragacanth, calcium phosphate, sucrose, mannitol, sorbitol, and dextrose.

Examples of the lubricant include, but are not limited to, magnesium stearate, stearic acid, and calcium stearate.

Examples of the disintegrant include, but are not limited to, croscarmellose sodium, polyvinylpyrrolidone, polyvinylpolypyrrolidone, agar, alginic acid or a salt thereof such as sodium alginate, sodium starch glycolate, and starch,

Examples of the glidant include, but are not limited to, colloidal silica, talc, cornstarch, and syloid.

Examples of the retention-enhancing excipient include, but are not limited to, bioadhesive polymers, mucoadhesive polymers, swelling hydrogels, and viscogenic agents. In one particular embodiment, such a retention-enhancing excipient is a carboxyvinyl polymer. Optionally, such a retention-enhancing excipient is a form of cellulose such as methyl cellulose, hydroxypropyl methylcellulose and/or Polycarbophil.

The drug may be made as a drug core (or tablet blend) with or without seal coating first and then enteric-coated with the pH-sensitive enteric coating material to produce a tablet. The drug core contains the drug, optionally contains one or more expient, and optionally further contains contain gum arabic, talc, polyvinyl pyrrolidone, carbopol gel, polyethylene glycol, titanium dioxide, lacquer solutions, and/or suitable organic solvents or solvent mixtures. These ingredients can be blended together and/or compressed to form the drug core or tablet blend. Optionally, dyestuffs or pigments may be added to the tablet or drug core for identification or to characterize different combinations of active compound doses.

Alternatively, the pharmaceutical composition may be administered using controlled release dosage forms. Controlled release within the scope of this invention can be taken to mean any one of a number of extended release dosage forms.

According to the present invention, the pharmaceutical composition is preferably administered orally to a host in need thereof. Optionally, the pharmaceutical composition may be administered or coadministered parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally.

The pharmaceutical composition of the present invention may be administered in conjunction with other agents for various purposes, such as to enhance the therapeutic efficacy, to increase the therapeutic index, and to reduce the side effects of the pharmaceutical composition.

For example, the pharmaceutical composition may be administered with various agents to reduce acid concentration in the stomach, such as an H2 inhibitor (e.g., cimetidine and ranitidine), an acid neutralizer (e.g., calcium carbonate), or a proton pump inhibitor (e.g., omeprazole, esomeprazole, lansoprazole, pantoprazole and rabeprazole).

A wide variety of anti-neoplastic agents may be used in conjunction with the pharmaceutical composition of the present invention for treating various diseases associated with abnormal cell proliferation such as cancer, such as antibiotic agents, antimetabolic agents, plant-derived agents, hormonal agents, biologic agents. The particular anti-neoplastic agent(s) used in conjunction with the pharmaceutical formulation may depend on the particular type of cancer to be treated.

These formulations can be used to treat a wide variety of diseases or conditions, such hematological disorders, benign tumors, cancer, restenosis, inflammatory diseases and autoimmune diseases.

### BRIEF DESCRIPTION OF THE FIGURES

FIG. 1 illustrates an anatomy of human intestines.

FIG. 2 is a graph showing changes in bioavailability after administration of pentostatin in buffered solution.

FIG. 3 are pharmacokinetic profiles of pentostatin after intravenous administration presented in both log and natural scales and representing the average values for each time point.

FIG. 4 are pharmacokinetic profiles of pentostatin after oral administration presented in both log and natural scales and representing the average values for each time point.

FIG. 5 are pharmacokinetic profiles of pentostatin after jejunal administration presented in both log and natural scales and representing the average values for each time point.

FIG. 6 are pharmacokinetic profiles of pentostatin after ileum administration presented in both log and natural scales and representing the average values for each time point.

FIG. 7 are pharmacokinetic profiles of pentostatin after intracolon administration presented in both log and natural scales and representing the average values for each time point.

FIG. 8 is a graph showing the relationship of dose of decitabine versus average values of area under the curve (AUC) for intravenous dosing

FIG. 9 are pharmacokinetic profiles of decitabine in systemic and portal vein after intravenous administration (IV) at a low dose (0.75 mg/kg) presented in a natural scale and representing the average values for each time point.

FIG. 10 are pharmacokinetic profiles of decitabine in systemic and portal vein after intravenous administration (IV) at a medium dose (1.5 mg/kg) presented in a natural scale and representing the average values for each time point.

FIG. 11 are pharmacokinetic profiles of decitabine in systemic and portal vein after intravenous administration (IV) at a high dose (2.5 mg/kg) presented in a natural scale and representing the average values for each time point.

FIG. 12 are pharmacokinetic profiles of decitabine in systemic and portal vein after portal vein (PV) administration at a high dose (2.5 mg/kg) presented in a natural scale and representing the average values for each time point.

FIG. 13 are pharmacokinetic profiles of decitabine in systemic and portal vein it after peroral (PO) administration at a high dose (2.5 mg/kg) presented in a natural scale and representing the average values for each time point.

FIG. 14 are pharmacokinetic profiles of decitabine in systemic and portal vein after local administration in the upper small intestine (USI) at a high dose (2.5 mg/kg) presented in a natural scale and representing the average values for each time point.

FIG. 15 are pharmacokinetic profiles of decitabine in systemic and portal vein after local administration in the lower small intestine (LSI) at a high dose (2.5 mg/kg) presented in a natural scale and representing the average values for each time point.

FIG. 16 are pharmacokinetic profiles of decitabine in systemic and portal vein after local administration in the colon (IC) at a high dose (2.5 mg/kg) presented in a natural scale and representing the average values for each time point for animal RS-44.

FIG. 17 are pharmacokinetic profiles of decitabine in systemic and portal vein after local administration in the colon (IC) at a high dose (2.5 mg/kg) presented in a natural scale and representing the average values for each time point for animal RS-45.

FIG. 18 is a pharmacokinetic profile of 9-nitro-camptothecin (9-NC) and 9-amino-camptothecin (9-AC) after administration of 0.2 mg/kg of 9-NC via the jejunal port.

FIG. 19 is a pharmacokinetic profile of 9-NC and 9-AC after administration of 0.2 mg/kg of 9-NC via the ileum port.

FIG. 20 is a pharmacokinetic profile of 9-NC and 9-AC after administration of 0.2 mg/kg of 9-NC via the colon port.

### DETAILED DESCRIPTION OF THE INVENTION

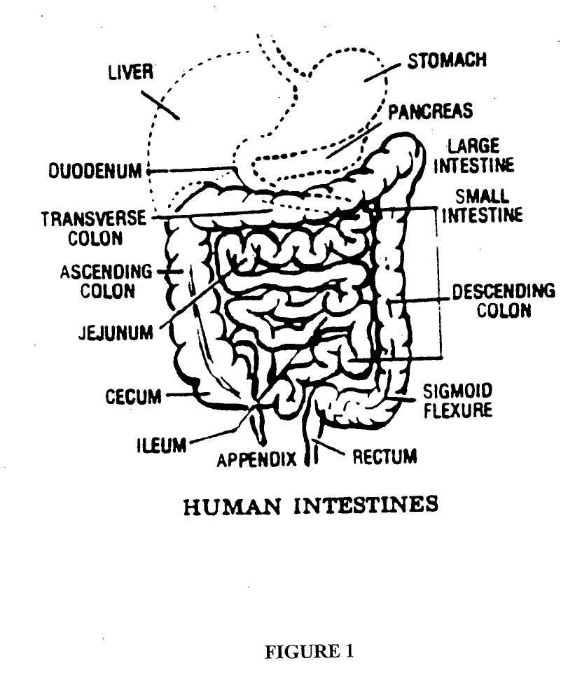

The present invention provides novel pharmaceutical formulations of drugs that can be orally delivered to a patient with enhanced bioavailability. Conventionally, for those drugs that are water-soluble and acid-labile parenteral administration is the only choice. As disclosed in the present invention, surprisingly, animals studies of the bioavailability of such drugs revealed that these drugs were preferably absorbed in specific regions of the gastrointestinal (GI) tract, such as the upper region of the intestine, the jejunum (FIG. 1). According to the present invention, oral formulations are provided for these drugs by specifically targeting this region of the GI tract where the drugs are preferably absorbed, thus bypassing the gastric degradation and significantly enhancing their oral bioavailability. In one aspect of the invention, the drug is formulated in the form of a tablet or capsule having an enteric coating that is resistant to gastric degradation at low acidic pH, but disintegrates when the pH in the GI tract increases to a threshold value, such as that of jejunum (about pH 5-7). The inventive formulation may further include an excipient that serves to increase the retention time of the drug in the upper small intestine, thereby maximizing the absorption of the drug into this particular region of the GI tract. In addition, drugs with poor oral bioavailability such as camptothecin compounds (e.g., 9-nitro-camptothecin) can also be formulated using similar strategies in order to significantly improve their bioavailability. These formulations can be used to treat a wide variety of diseases or conditions, such hematological disorders, benign tumors, cancer, restenosis, and inflammatory diseases.

1. Pharmaceutical Compositions of the Present Invention

In one aspect of the invention, a pharmaceutical composition is provided. The pharmaceutical composition comprises: a water-soluble, acid-labile drug enteric-coated with a coating material that dissolves at pH above about 5.2.

According to the invention, the solubility of the drug is preferably above 1 mg/ml in water or aqueous solution, more preferably above 5 mg/ml in water or aqueous solution, and most preferably above 10 mg/ml in water or aqueous solution.

Also according to the invention, the drug is labile preferably at pH lower than 5.0, more preferably at pH lower than 4.0, and most preferably at pH lower than 2.0. It is known that the gastric juice has a pH about 1.2. Thus, drugs that are soluble in gastric juice but labile under such an acidic environment are preferably included within the scope of the invention.

Examples of the drug includes, but are not limited to, cytidine analogs or derivatives such as 5-azacytidine and 5-aza-2′-deoxycytidine (5-aza-CdR or decitabine), and 2′-deoxyadenosine analogs and derivatives such as 2′-deoxycoformycin (also referred to as dCF, pentostatin, or NIPENT®), fludarabine monophosphate (FLU), and 2-chloro-2′-deoxyadenosine (also known as cladribine or 2CDA).

The coating material for enteric-coating of the drug is pH-sensitive and preferably or selectively dissolves at a threshold pH above about 5.2, optionally at pH above about 5.5, optionally at pH above about 5.8, optionally at pH above about 6.0, optionally at pH above about 6.2, optionally at pH above about 6.5, optionally at pH above about 6.5, and most preferably at pH above about 6.8, optionally at pH above about 7.0, optionally at pH above about 7.2, or optionally at pH above about 7.5. The pharmaceutical composition is preferred to substantially disintegrate in an aqueous medium at a pH equal or above the threshold pH within 3 hours, optionally within 2 hours, optionally within 1 hour, more preferably within 30 min, and most preferably within 15 min. The pharmaceutical composition is considered to be substantially disintegrated if at least 50% of the composition disintegrates, e.g., undergoes rupture.

This formulation is believed to protect the drug from decomposition in the gastric juice in the stomach and selectively release the drug in the upper region of the small intestine, preferably in the jejunum, where the pH is slightly acid and close to neutral, which is beyond the threshold pH of the enteric-coat. The disintegration of the enteric-coat leads to selective release of the drug at the specific site of the GI tract where the drug is preferably absorbed, thereby enhancing the oral bioavailability of the drug. In addition, by bypassing decomposition in the stomach, side effects such as damages to the gastric mucosa by the drug and nausea due to stomach irritation can be avoided.

Examples of such a coating material include, but are not limited to, cellulose phthalates (e.g, hydropropylmethylcellulose phthalates (HPMCPs)) that selectively dissolve at pH above 5.6, the Eudragit® family of polymers which are anionic polymer based on methacrylic acid and methacrylates with carboxyl functional groups (e.g., Eudragit L30D with threshold pH of 5.6, Eudragit L with threshold pH of 6.0, and Eudragit S with threshold pH of 6.8), Aquateric with threshold pH of 5.8, polyvinylacetate phthalate (PVAP) that releases drug at pH values above about 5.0, Shellac® that is obtained from a gummy exudation produced by female insects, Laccifer lacca kerr, and releases drug at about pH7.0, and cellulose acetate phthalate (CAP) with threshold pH of 6.0.

In a preferred embodiment, the drug is enteric-coated with Eudragit L100 with threshold pH of 6.0 or L-100-55 with a threshold pH of 5.5.

Also according to the invention, the pharmaceutical composition is preferred not to substantially disintegrate in an acidic, aqueous medium at pH 1.0-3.0 for at least 1 hour, more preferred not to substantially disintegrate in an acidic, aqueous medium at pH 1.2-2.0 for at least 1 hour, more preferably for at least 2 hours, and most preferably for at least 3 hours. Optionally, the pharmaceutical formulation does not substantially disintegrate in an acidic, aqueous medium at pH 1.2-1.5 for at least 1 hour, more preferably for at least 2 hours, and most preferably for at least 3 hours. The composition is considered to be substantially disintegrated if at least 50% of the composition disintegrates, e.g., undergoes rupture.

In addition, the pharmaceutical composition preferably disintegrates substantially in an aqueous medium at pH 5.2-7.5 within 1 hour, more preferably disintegrates substantially in an aqueous medium at pH 6.0-7.2 within 30 minutes, and most preferably disintegrates substantially in an aqueous medium at pH 6.5-7.0 within 15 minutes.

The amount of the enteric-coating material is preferably 1-10% w/w in the composition, more preferably 2-8% w/w in the composition, and most preferably 3-6% w/w in the composition.

The pharmaceutical composition may be in a form of tablet or capsule. In a preferred embodiment, the composition is in a form of tablet. The hardness of the tablet without the enteric-coat is preferably at least 4 kp, more preferably at least 8 kp, and most preferably 10 kp. The size of the tablet is preferably 5-20 mm, more preferably 8-15 mm, and most preferably 10-13 mm.

In any of the above dosage forms, the concentration of the drug is preferably 0.1-20% w/w, more preferably 1-10% w/w, and most preferably 2-5% w/w.

Optionally, the pharmaceutical composition may further comprise a seal-coating material that seals the drug to prevent decomposition due to exposure to moisture, such as hydroxy propylmethylcellulose. Accordingly, the core of the drug is first sealed by the seal-coating material and then coated with the enteric-coating material. This is particularly useful for the formulation of decitabine which is prone to decomposition in exposure to moisture.

Optionally, the pharmaceutical composition may further comprise buffer salt such as potassium or sodium phosphate in an amount sufficient to maintain the pH of the local environment to be 5.2-7.0 when the pharmaceutical composition is dissolved in the GI tract. Examples of such buffer salts include, but are not limited to, KH₂PO₄ and Na₂HPO₄. This formulation is particularly useful for oral formulation of pentostatin since it was discovered that there was a significant increase in oral bioavailability of pentostatin from the jejunum when pentostatin was administered as a pH7-buffered solution as compared to that in normal saline.

According to the invention, another pharmaceutical composition is provided. The pharmaceutical composition comprises: a camptothecin compound enteric-coated with a coating material that dissolves at pH above 5.2.

The coating material for enteric-coating of the camptothecin compound is pH-sensitive and preferably or selectively dissolves at pH above about 5.2, preferably at pH above about 5.8, more preferably at pH above about 6.0, and most preferably at pH above about 6.4.

Examples of such a coating material include, but are not limited to, cellulose phthalates (e.g, hydropropylmethylcellulose phthalates (HPMCPs)) that selectively dissolve at pH above 5.6, the Eudragit® family of polymers which are anionic polymer based on methacrylic acid and methacrylates with carboxyl functional groups (e.g., Eudragit L30D with threshold pH of 5.6, Eudragit L with threshold pH of 6.0, and Eudragit S with threshold pH of 6.8), Aquateric with threshold pH of 5.8, polyvinylacetate phthalate (PVAP) that releases drug at pH values above about 5.0, Shellac® that is obtained from a gummy exudation produced by female insects, Laccifer lacca kerr, and releases drug at about pH7.0, and cellulose acetate phthalate (CAP) with threshold pH of 6.0.

In a preferred embodiment, the drug is enteric-coated with Eudragit L100 with threshold pH of 6.0 or L-100-55 with a threshold pH of 5.5.

The camptothecin compound may be the original 20(S)-camptothecin isolated from the plant, Camptotheca acuminata, analogs of 20(S)-camptothecin, derivatives of 20(S)-camptothecin, prodrugs of 20(S)-camptothecin, and pharmaceutically active metabolites of 20(S)-camptothecin.

Examples of camptothecin derivatives include, but are not limited to, 9-nitro-20(S)-camptothecin, 9-amino-20(S)-camptothecin, 9-methyl-camptothecin, 9-chloro-camptothecin, 9-flouro-camptothecin, 7-ethyl camptothecin, 10-methyl-camptothecin, 10-chloro-camptothecin, 10-bromo-camptothecin, 10-fluoro-camptothecin, 9-methoxy-camptothecin, 11-fluoro-camptothecin, 7-ethyl-10-hydroxy camptothecin, 10,11-methylenedioxy camptothecin, and 10,11-ethylenedioxy camptothecin, and 7-(4-methylpiperazinomethylene)-10,11-methylenedioxy camptothecin. Prodrugs of camptothecin include, but are not limited to, esterified camptothecin derivatives as decribed in U.S. Pat. No. 5,731,316, such as camptothecin 20-O-propionate, camptothecin 20-O-butyrate, camptothecin 20-O-valerate, camptothecin 20-O-heptanoate, camptothecin 20-O-nonanoate, camptothecin 20-O-crotonate, camptothecin 20-O-2′,3′-epoxy-butyrate, nitrocamptothecin 20-O-acetate, nitrocamptothecin 20-O-propionate, and nitrocamptothecin 20-O-butyrate.

In particular, when substituted camptothecins are used, a large range of substitutions may be made to the camptothecin scaffold, while still retaining activity. In a preferable embodiment, the camptothecin scaffold is substituted at the 7, 9, 10, 11, and/or 12 positions. Such preferable substitutions may serve to provide differential activities over the unsubstituted camptothecin compound. Especially preferable are 9-nitrocamptothecin, 9-aminocamptothecin, 10,11-methylendioxy-20(S)-camptothecin, topotecan, irinotecan, 7-ethyl-10-hydroxy camptothecin, or another substituted camptothecin that is substituted at least one of the 7, 9, 10, 11, or 12 positions.

According to the invention, the camptothecin compound is preferably a water-insoluble camptothecin compound such as 9-nitrocamptothecin and 9-aminocamptothecin. It is believed that the oral bioavailability of these camptothecin compounds can be improved by selectively delivering the drugs to the upper region of the small intestine, e.g., the jejunum. In addition, since these camptothecin compounds are resistant to decomposition in exposure to moisture, they may be formulated by directly enteric-coating without seal-coating in between the drug core and the enteric-coat.

In any of the above dosage forms, the concentration of the camptothecin compound is preferably 0.01-20% w/w, more preferably 0.1-10% w/w, and most preferably 0.2-5% w/w.

According to any of the above embodiments of the invention, the pharmaceutical composition may further comprise one or more pharmaceutically acceptable excipient. The excipient may be a diluent, lubricant, disintegrant, glidant, and/or an excipient that serves to increase the retention time in the upper small intestine (hereinafter referred to as the “retention-enhancing excipient”).

Examples of the diluent include, but are not limited to, microcrystalline cellulose (e.g., Avicel PH102®), lactose monohydrate (e.g., Fast Flo lactose 316®), starch (e.g., Starch 1500®, maize starch, wheat starch, rice starch, and potato starch), gelatin, gum, tragacanth, calcium phosphate, sucrose, mannitol, sorbitol, and dextrose.

Examples of the lubricant include, but are not limited to, magnesium stearate, stearic acid, and calcium stearate.

Examples of the disintegrant include, but are not limited to, croscarmellose sodium, polyvinylpyrrolidone, polyvinylpolypyrrolidone, agar, alginic acid or a salt thereof such as sodium alginate, sodium starch glycolate, and starch,

Examples of the glidant include, but are not limited to, colloidal silica, talc, cornstarch, and syloid.

Examples of the retention-enhancing excipient include, but are not limited to, bioadhesive polymers, mucoadhesive polymers, swelling hydrogels, and viscogenic agents. In one particular embodiment, such a retention-enhancing excipient is a carboxyvinyl polymer (Carbomer 934P). Optionally, such a retention-enhancing excipient is a form of cellulose such as methyl cellulose, hydroxypropyl methylcellulose (HPMC) and/or Polycarbophil.

The drug may be made as a drug core core (or tablet blend) with or without seal coating first and then enteric coated with the pH-sensitive enteric coating material to produce a tablet. The drug core contains the drug, optionally contains one or more excipient, and optionally further contains gum arabic, talc, polyvinyl pyrrolidone, carbopol gel, polyethylene glycol, titanium dioxide, lacquer solutions, and/or suitable organic solvents or solvent mixtures. These ingredients can be blended together and/or compressed to form the drug core or tablet blend, and optionally, there is no additional polymer-barrier (other than a seal coating which is applied when it is necessary to prevent moisture infusion into the core) in between the core and enteric coat. Upon administration of the composition to the GI tract, the drug is delivered to the specific GI region reaching a pH threshold at which the pH-sensitive enteric coating material disintegrates and releases the drug to this particular GI region (e.g., the jejunum) in a relatively short period of time, e.g., within 1-3 hours of delivery. The relatively fast disintegration of the composition in the GI tract may cause an excessively rapid release of the drug, the so-called dose-dumping effect. In the present invention, the dose-dumping effect may be considered to be desirable because the retention time of a composition in the upper region of the small intestine can be short (e.g., 3±1 hr) and constant, irrespective of the fed and fasted state of the subject (Davis et al. (1984) Gut 27, pp886), and fast dissolution and thus dose-dumping would allow maximum absorption of the drug into the plasma, thereby enhancing the oral bioavailability of the drug.

Optionally, dyestuffs or pigments may be added to the tablet or drug core for identification or to characterize different combinations of active compound doses.

Alternatively, the pharmaceutical composition may be administered using controlled release dosage forms. Controlled release within the scope of this invention can be taken to mean any one of a number of extended release dosage forms.

The following terms may be considered to be substantially equivalent to controlled release, for the purposes of the present invention: continuous release, controlled release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.).

According to the present invention, the pharmaceutical composition is preferably administered orally to a host in need thereof. Optionally, the pharmaceutical composition may be administered or coadministered parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally.

The pharmaceutical composition of the present invention may be used in the form of kits. The arrangement and construction of such kits is conventionally known to one of skill in the art. Such a kit may include containers for containing the inventive composition, and/or other apparatus for administering the inventive composition.

The kit may optionally further include instructions. The instructions may describe how to administer the pharmaceutical formulation to a patient. It is noted that the instructions may optionally describe the administration methods according to the present invention.

2. Combination Therapy Using the Pharmaceutical Composition

The pharmaceutical composition of the present invention may be administered in conjunction with other agents for various purposes, such as to enhance the therapeutic efficacy, to increase the therapeutic index, and to reduce the side effects of the pharmaceutical composition.

For example, the pharmaceutical composition may be administered with various agents to reduce acid concentration in the stomach. This reduces acid lability and allows for enhanced concentrations of the drug for enhanced gastric and/or intestinal absorption. For example, the adenosine analog may be co-administered with an H2 inhibitor such as cimetidine and ranitidine, an acid neutralizer such as calcium carbonate, or a proton pump inhibitor (e.g., omeprazole, esomeprazole, lansoprazole, pantoprazole and rabeprazole).

Furthermore, the pharmaceutical composition may be co-administered using a dosage form that reduces the effect of acid lability on their bioavailability. Co-administration in the context of this invention may be defined to mean the administration of more than one therapeutic in the course of a coordinated treatment to achieve an improved clinical outcome. Such co-administration may also be coextensive, that is, occurring during overlapping periods of time.

A wide variety of anti-neoplastic agents may be used in conjunction with the pharmaceutical composition of the present invention for treating various diseases associated with abnormal cell proliferation such as cancer. The particular anti-neoplastic agent(s) used in conjunction with the pharmaceutical formulation may depend on the particular type of cancer to be treated.

The antineoplastic agent may be an antibiotic agent. Antibiotic agents are a group of anticancer drugs that are produced in a manner similar to antibiotics by a modification of natural products. Examples of antibiotic agents include, but are not limited to, anthracyclines (e.g. doxorubicin, daunorubicin, epirubicin, idarubicin and anthracenedione), mitomycin C, bleomycin, dactinomycin, plicatomycin. These antibiotic agents interfere with cell growth by targeting different cellular components. For example, anthracyclines are generally believed to interfere with the action of DNA topoisomerase II in the regions of transcriptionally active DNA, which leads to DNA strand scissions. Bleomycin is generally believed to chelate iron and form an activated complex, which then binds to bases of DNA, causing strand scissions and cell death. A combination therapy of an antibiotic agent and the pharmaceutical formulation of the present invention may have therapeutic synergistic effects on cancer and reduce sides affects associated with these chemotherapeutic agents.

The antineoplastic agent may be an antimetabolic agent. Antimetabolic agents are a group of drugs that interfere with metabolic processes vital to the physiology and proliferation of cancer cells. Actively proliferating cancer cells require continuous synthesis of large quantities of nucleic acids, proteins, lipids, and other vital cellular constituents. Many of the antimetabolites inhibit the synthesis of purine or pyrimidine nucleosides or inhibit the enzymes of DNA replication. Some antimetabolites also interfere with the synthesis of ribonucleosides and RNA and/or amino acid metabolism and protein synthesis as well. By interfering with the synthesis of vital cellular constituents, antimetabolites can delay or arrest the growth of cancer cells. Examples of antimetabolic agents include, but are not limited to, fluorouracil (5-FU), floxuridine (5-FUdR), methotrexate, leucovorin, hydroxyurea, thioguanine (6-TG), mercaptopurine (6-MP), cytarabine, fludarabine phosphate, cladribine (2-CDA), asparaginase, and gemcitabine. A combination therapy of an antimetabolic agent and the pharmaceutical formulation of the present invention may have therapeutic synergistic effects on cancer and reduce sides affects associated with these chemotherapeutic agents.

The antineoplastic agent may also be a plant-derived agent. Plant-derived agents are a group of drugs that are derived from plants or modified based on the molecular structure of the agents. Examples of plant-derived agents include, but are not limited to, vinca alkaloids (e.g., vincristine, vinblastine, vindesine, vinzolidine and vinorelbine), podophyllotoxins (e.g., etoposide (VP-16) and teniposide (VM-26)), taxanes (e.g., paclitaxel and docetaxel). These plant-derived agents generally act as antimitotic agents that bind to tubulin and inhibit mitosis. Podophyllotoxins such as etoposide are believed to interfere with DNA synthesis by interacting with topoisomerase II, leading to DNA strand scission. A combination therapy of a plant-derived agent and the pharmaceutical formulation of the present invention may have therapeutic synergistic effects on cancer and reduce side affects associated with these chemotherapeutic agents.

The antineoplastic agent may be a hormonal agent. The hormonal agents are a group of drug that regulate the growth and development of their target organs. Most of the hormonal agents are sex steroids and their derivatives and analogs thereof, such as estrogens, androgens, and progestins. These hormonal agents may serve as antagonists of receptors for the sex steroids to down regulate receptor expression and transcription of vital genes. Examples of such hormnonal agents are synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone.

The antineoplastic agent may be a biologic agent. Biologic agents are a group of biomolecules that elicit cancer/tumor regression when used alone or in combination with chemotherapy and/or radiotherapy. Examples of biologic agents include, but are not limited to, immuno-modulating proteins such as cytokines, monoclonal antibodies against tumor antigens, tumor suppressor genes, and cancer vaccines. Combination therapy of the biologic agent and the pharmaceutical formulation of the present invention may have therapeutic synergistic effects on cancer, enhance the patient's immune responses to tumorigenic signals, and reduce potential sides affects associated with this biologic agent.

Cytokines possess profound immunomodulatory activity. Some cytokines such as interleukin-2 (IL-2, aldesleukin) and interferon-α (IFN-α) demonstrate antitumor activity and have been approved for the treatment of patients with metastatic renal cell carcinoma and metastatic malignant melanoma. IL-2 is a T-cell growth factor that is central to T-cell-mediated immune responses. The selective antitumor effects of IL-2 on some patients are believed to be the result of a cell-mediated immune response that discriminate between self and non-self. Examples of interleukins that may be used in conjunction with the pharmaceutical formulation of the present invention include, but are not limited to, interleukin 2 (IL-2), and interleukin 4 (IL-4), interleukin 12 (IL-12).

Interferon-α includes more than 23 related subtypes with overlapping activities, all of the IFN-α subtypes within the scope of the present invention. IFN-α has demonstrated activity against many solid and hematologic malignancies, the later appearing to be particularly sensitive. Examples of interferons that may be used in conjunction with the TNF mutein of the present invention, but are not limited to, interferon-α, interferon-β (fibroblast interferon) and interferon-γ (fibroblast interferon).

Other cytokines that may be used in conjunction with the pharmaceutical formulation of the present invention include those cytokines that exert profound effects on hematopoiesis and immune functions. Examples of such cytokines include, but are not limited to erythropoietin (epoietin-α), granulocyte-CSF (filgrastin), and granulocyte, macrophage-CSF (sargramostim). These cytokines may be used in conjunction with the TNF mutein of the present invention to reduce chemotherapy-induced myelopoietic toxicity.

Immuno-modulating agents other than cytokines may also be used in conjunction with the TNF mutein of the present invention to inhibit abnormal cell growth. Examples of such immuno-modulating agents include, but are not limited to bacillus Calmette-Guerin, levamisole, and octreotide, a long-acting octapeptide that mimics the effects of the naturally occurring hormone somatostatin.

Monoclonal antibodies against tumor antigens are antibodies elicited against antigens expressed by tumors, preferably tumor-specific antigens. For example, monoclonal antibody HERCEPTIN® (Trastruzumab) is raised against human epidermal growth factor receptor2 (HER2) that is overexpressed in some breast tumors including metastatic breast cancer. Overexpression of HER2 protein is associated with more aggressive disease and poorer prognosis in the clinic. HERCEPTIN® is used as a single agent for the treatment of patients with metastatic breast cancer whose tumors over express the HER2 protein. Combination therapy including the pharmaceutical formulation of the present invention and HERCEPTIN® may have therapeutic synergistic effects on tumors, especially on metastatic cancers.

Another example of monoclonal antibodies against tumor antigens is RITUXAN® (Rituximab) that is raised against CD20 on lymphoma cells and selectively deplete normal and maligant CD20⁺ pre-B and mature B cells. RITUXAN® is used as single agent for the treatment of patients with relapsed or refractory low-grade or follicular, CD20+, B cell non-Hodgkin's lymphoma. Combination therapy including the pharmaceutical formulation of the present invention and RITUXAN® may have therapeutic synergistic effects not only on lymphoma, but also on other forms or types of malignant tumors.

Other examples of anti-cancer antibodies on the market or in the process of the FDA approval and may be used in combination with CPT and a COX-2 inhibitor include, but are not limited to, MYLOTARG® (gemtuzumab ozogamicin) which is an monoclonal antibody approved for treating acute myeloid leukemia (AML), CAMPATH® (alemtuzumab) for B cell chronic lymphocytic leukemia, ZEVALIN® (ibritumomab yiuxetan) for non-Hodgkin's lymphoma (NHL), PANOREX® (edrecolomab) for colorectal cancer, BEXXAR® (tositumomab) for treating NHL, ERBITUX® (cetuximab) which is a monoclonal antibody targeting epidermal growth factor (EGF) and for treating various cancers, AVASTIN® (bevacizumab) which is a monoclonal antibody targeting vascular endothelial growth factor (VEGF) and for treating various cancers, and pemtumomab for treating ovarian cancer.

Tumor suppressor genes are genes that function to inhibit the cell growth and division cycles, thus preventing the development of neoplasia. Mutations in tumor suppressor genes cause the cell to ignore one or more of the components of the network of inhibitory signals, overcoming the cell cycle check points and resulting in a higher rate of controlled cell growth-cancer. Examples of the tumor suppressor genes include, but are not limited to, DPC-4, NF-1, NF-2, RB, p53, WT1, BRCA1 and BRCA2. The pharmaceutical formulation of the present invention may be used in combination with a therapy delivering the tumor suppressor in vivo (e.g., via gene therapy) to treat various forms of cancer.

The inventive combination of therapeutic agents may be used in the form of kits. The arrangement and construction of such kits is conventionally known to one of skill in the art. Such kits may include containers for containing the inventive combination of therapeutic agents and/or compositions, and/or other apparatus for administering the inventive combination of therapeutic agents and/or compositions.

3. Indications that May be Treated With the Pharmaceutical Composition

Preferable indications that may be treated using the pharmaceutical compositions of the present invention include those involving undesirable or uncontrolled cell proliferation. Such indications include benign tumors, various types of cancers such as primary tumors and tumor metastasis, hematological disorders (e.g. leukemia, myelodysplastic syndrome and sickle cell anemia), restenosis (e.g. coronary, carotid, and cerebral lesions), abnormal stimulation of endothelial cells (arteriosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

Generally, cells in a benign tumor retain their differentiated features and do not divide in a completely uncontrolled manner. A benign tumor is usually localized and nonmetastatic. Specific types benign tumors that can be treated using the present invention include hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

In a malignant tumor cells become undifferentiated, do not respond to the body's growth control signals, and multiply in an uncontrolled manner. The malignant tumor is invasive and capable of spreading to distant sites (metastasizing). Malignant tumors are generally divided into two categories: primary and secondary. Primary tumors arise directly from the tissue in which they are found. A secondary tumor, or metastasis, is a tumor which is originated elsewhere in the body but has now spread to a distant organ. The common routes for metastasis are direct growth into adjacent structures, spread through the vascular or lymphatic systems, and tracking along tissue planes and body spaces (peritoneal fluid, cerebrospinal fluid, etc.)

Specific types of cancers or malignant tumors, either primary or secondary, that can be treated using this invention include leukemia, breast cancer, skin cancer, bone cancer, prostate cancer, liver cancer, lung cancer, brain cancer, cancer of the larynx, gall bladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuronmas, intestinal ganglioneuromas, hyperplastic corneal nerve tumor, marfanoid habitus tumor, Wilm's tumor, seminoma, ovarian tumor, leiomyoma tumor, cervical dysplasia and in situ carcinoma, neuroblastoma, retinoblastoma, medulloblastoma, soft tissue sarcoma, malignant carcinoid, topical skin lesion, mycosis fungoides, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, epidermoid carcinomas, and other carcinomas and sarcomas.

Hematologic disorders include abnormal growth of blood cells which can lead to dysplastic changes in blood cells and hematologic malignancies such as various leukemias. Examples of hematologic disorders include but are not limited to acute myeloid leukemia, acute promyelocytic leukemia, acute lymphoblastic leukemia, chronic myelogenous leukemia, the myelodysplastic syndromes, and sickle cell anemia.

Acute myeloid leukemia (AML) is the most common type of acute leukemia that occurs in adults. Several inherited genetic disorders and immunodeficiency states are associated with an increased risk of AML. These include disorders with defects in DNA stability, leading to random chormosomal breakage, such as Bloom's syndrome, Fanconi's anemia, Li-Fraumeni kindreds, ataxia-telangiectasia, and X-linked agammaglobulinemia.

Acute promyelocytic leukemia (APML) represents a distinct subgroup of AML. This subtype is characterized by promyelocytic blasts containing the 15; 17 chromosomal translocation. This translocation leads to the generation of the fusion transcript comprised of the retinoic acid receptor and a sequence PML.

Acute lymphoblastic leukemia (ALL) is a heterogenerous disease with distinct clinical features displayed by various subtypes. Reoccurring cytogenetic abnormalities have been demonstrated in ALL. The most common cytogenetic abnormality is the 9;22 translocation. The resultant Philadelphia chromosome represents poor prognosis of the patient.

Chronic myelogenous leukemia (CML) is a clonal myeloproliferative disorder of a pluripotent stem cell. CML is characterized by a specific chromosomal abnormality involving the translocation of chromosomes 9 and 22, creating the Philadelphia chromosome. Ionizing radiation is associated with the development of CML.

The myelodysplastic syndromes (MDS) are heterogeneous clonal hematopoietic stem cell disorders grouped together because of the presence of dysplastic changes in one or more of the hematopoietic lineages including dysplastic changes in the myeloid, erythroid, and megakaryocytic series. These changes result in cytopenias in one or more of the three lineages. Patients afflicted with MDS typically develop complications related to anemia, neutropenia (infections), or thrombocytopenia (bleeding). Generally, from about 10% to about 70% of patients with MDS develop acute leukemia.

Treatment of abnormal cell proliferation due to insults to body tissue during surgery may be possible for a variety of surgical procedures, including joint surgery, bowel surgery, and cheloid scarring. Diseases that produce fibrotic tissue include emphysema. Repetitive motion disorders that may be treated using the present invention include carpal tunnel syndrome. An example of cell proliferative disorders that may be treated using the invention is a bone tumor.

The proliferative responses associated with organ transplantation that may be treated using this invention include those proliferative responses contributing to potential organ rejections or associated complications. Specifically, these proliferative responses may occur during transplantation of the heart, lung, liver, kidney, and other body organs or organ systems.

Abnormal angiogenesis that may be may be treated using this invention include those abnormal angiogenesis accompanying rheumatoid arthritis, ischemic-reperfusion related brain edema and injury, cortical ischemia, ovarian hyperplasia and hypervascularity, (polycystic ovary syndrom), endometriosis, psoriasis, diabetic retinopaphy, and other ocular angiogenic diseases such as retinopathy of prematurity (retrolental fibroplastic), macular degeneration, corneal graft rejection, neuroscular glaucoma and Oster Webber syndrome.

Diseases associated with abnormal angiogenesis require or induce vascular growth. For example, corneal angiogenesis involves three phases: a pre-vascular latent period, active neovascularization, and vascular maturation and regression. The identity and mechanim of various angiogenic factors, including elements of the inflammatory response, such as leukocytes, platelets, cytokines, and eicosanoids, or unidentified plasma constituents have yet to be revealed.

The pharmaceutical composition of the present invention may also be used for treating diseases associated with undesired or abnormal angiogenesis alone or in conjunction with an anti-angiogenesis agent.

The particular dosage of these agents required to inhibit angiogenesis and/or angiogenic diseases may depend on the severity of the condition, the route of administration, and related factors that can be decided by the attending physician. Generally, accepted and effective daily doses are the amount sufficient to effectively inhibit angiogenesis and/or angiogenic diseases.

According to this embodiment, the pharmaceutical composition of the present invention may be used to treat a variety of diseases associated with undesirable angiogenesis such as retinal/choroidal neuvascularization and corneal neovascularization. Examples of retinal/choroidal neuvascularization include, but are not limited to, Bests diseases, myopia, optic pits, Stargarts diseases, Pagets disease, vein occlusion, artery occlusion, sickle cell anemia, sarcoid, syphilis, pseudoxanthoma elasticum carotid abostructive diseases, chronic uveitis/vitritis, mycobacterial infections, Lyme's disese, systemic lupus erythematosis, retinopathy of prematurity, Eales disease, diabetic retinopathy, macular degeneration, Bechets diseases, infections causing a retinitis or chroiditis, presumed ocular histoplasmosis, pars planitis, chronic retinal detachment, hyperviscosity syndromes, toxoplasmosis, trauma and post-laser complications, diseases associated with rubesis (neovascularization of the angle) and diseases caused by the abnormal proliferation of fibrovascular or fibrous tissue including all forms of proliferative vitreoretinopathy. Examples of comeal neuvascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, diabetic retinopathy, retinopathy of prematurity, corneal graft rejection, Mooren ulcer, Terrien's marginal degeneration, marginal keratolysis, polyarteritis, Wegener sarcoidosis, Scleritis, periphigoid radial keratotomy, neovascular glaucoma and retrolental fibroplasia, syphilis, Mycobacteria infections, lipid degeneration, chemical burns, bacterial ulcers, fungal ulcers, Herpes simplex infections, Herpes zoster infections, protozoan infections and Kaposi sarcoma.

The pharmaceutical composition of the present invention may be used for treating chronic inflammatory diseases associated with abnormal angiogenesis. The chronic inflammation depends on continuous formation of capillary sprouts to maintain an influx of inflammatory cells. The influx and presence of the inflammatory cells produce granulomas and thus, maintains the chronic inflammatory state. Inhibition of angiogenesis using the composition of the present invention may prevent the formation of the granulosmas, thereby alleviating the disease. Examples of chronic inflammatory disease include, but are not limited to, inflammatory bowel diseases such as Crohn's disease and ulcerative colitis, psoriasis, sarcoidois, and rhematoid arthritis.

Inflammatory bowel diseases such as Crohn's disease and ulcerative colitis are characterized by chronic inflammation and angiogenesis at various sites in the gastrointestinal tract. For example, Crohn's disease occurs as a chronic transmural inflammatory disease that most commonly affects the distal ileum and colon but may also occur in any part of the gastrointestinal tract from the mouth to the anus and perianal area. Patients with Crohn's disease generally have chronic diarrhea associated with abdominal pain, fever, anorexia, weight loss and abdominal swelling. Ulcerative colitis is also a chronic, nonspecific, inflammatory and ulcerative disease arising in the colonic mucosa and is characterized by the presence of bloody diarrhea. These inflammatory bowel diseases are generally caused by chronic granulomatous inflammation throughout the gastrointestinal tract, involving new capillary sprouts surrounded by a cylinder of inflammatory cells. Inhibition of angiogenesis by the composition of the present invention should inhibit the formation of the sprouts and prevent the formation of granulomas. The inflammatory bowel diseases also exhibit extra intestinal manifectations, such as skin lesions. Such lesions are characterized by inflammation and angiogenesis and can occur at many sites other the gastrointestinal tract. Inhibition of angiogenesis by the composition of the present invention should reduce the influx of inflammatory cells and prevent the lesion formation.

Sarcoidois, another chronic inflammatory disease, is characterized as a multisystem granulomatous disorder. The granulomas of this disease can form anywhere in the body and, thus, the symptoms depend on the site of the granulomas and whether the disease is active. The granulomas are created by the angiogenic capillary sprouts providing a constant supply of inflammatory cells. By using the composition of the present invention to inhibit angionesis, such granulomas formation can be inhibited. Psoriasis, also a chronic and recurrent inflammatory disease, is characterized by papules and plaques of various sizes. Treatment using the composition of the present invention should prevent the formation of new blood vessels necessary to maintain the characteristic lesions and provide the patient relief from the symptoms.

Rheumatoid arthritis (RA) is also a chronic inflammatory disease characterized by non-specific inflammation of the peripheral joints. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis. Treatment using the composition of the present invention alone or in conjunction with other anti-RA agents should prevent the formation of new blood vessels necessary to maintain the chronic inflammation and provide the RA patient relief from the symptoms.

The pharmaceutical composition of the present invention may also be used to treat autoimmune diseases. Autoimmune diseases refer to a wide range of degenerative diseases caused by the immune system attacking a person's own cells. Autoimmune diseases are usually classified clinically in a variety of ways. In light of affected parts by the diseases, there are, for example, degenerative diseases of supporting tissues and connective tissues; autoimmune degenerative diseases of salivary glands, particularly Sjogren's disease; autoimmune degenerative diseases of kidneys, particularly systemic lupus erythematodes (SLE) and glomerulonephritis; autoimmune degenerative diseases of joints, particularly rheumatoid arthritis; and autoimmune degenerative diseases of blood vessels such as generalized necrotizing angitis and granulomatous angitis; and multiple sclerosis. Alternatively, autoimmune diseases can be classified in one of the two different categories: cell-mediated disease (i.e. T-cell) or antibody mediated disorders. Examples of cell-mediated autoimmune diseases include multiple sclerosis, rheumatoid arthritis, autoimmune thyroiditis, and diabetes mellitus. Antibody-mediated autoimmune disorders include myasthenia gravis and SLE.

### EXAMPLE

1. Pharmacokinetics of Pentostatin in IVAP Beagle Dog Model

To determine whether pentostatin can be preferably absorbed in a specific region(s) of the GI tract with high bioavailability, pharmacokinetics studies of pentostatin were designed and performed as summarized in the following.

Pentostatin was administered to IVAP beagle dogs (n=3) at 0.2 mg/kg via intravenous, oral and through previously implanted intestinal ports (jejunum, ileum and colon). Extravascular administration of pentostatin was done in saline water for infusion or in a pH=7 phosphate buffer solution to control the intestinal pH. Blood samples were taken systemic and through a port in the portal vein, plasma separated and kept frozen at −20° C. until analysis.

It was discovered that bioavailability of pentostatin was site dependent and was increased by administration of the drug in a buffered solution. The highest bioavailability was achieved after administration in the jejunum (F=0.54 versus 0.88 with buffered solution). Absorption of pentostatin occurs in a preferential manner in the jejunum. Administration via jejunal port yielded the highest plasma concentration (Cmax) and AUC and the lowest variability.

Details of the animal experiments are described in the following.

1) Materials and Methods

Six adult male beagle dogs (obtained from Summit Ridge Farms, Susquehanna, Pa.; between the ages of 1 and 5 years and weighed between 9 and 16 kg) were prepared with indwelling catheters in the Upper and Lower Small Intestines (USI and LSI), Colon (IC) and Portal Vein (PV) and were dosed with pentostatin at 0.2 mg/kg. The drug formulation was delivered either orally, intraveneously or, in most cases, through the indwelling ports and blood samples were drawn to determine the pharmacokinetics of each permutation. Systemic blood samples were collected from acutely placed IV catheters in the brachial vein, while PV samples were collected through the indwelling port and catheter. All blood samples were spun down, the plasma decanted, frozen and stored at −70 deg Celsius before subject to analysis.

The IVAP dog is prepared with indwelling catheters attached to subcutaneous ports (Titanium Vascular Access Portsg from Access Technologies in Skokie, Ill.) for delivery of drug formulations directly to various parts of the Gastrointestinal Tract. Regional differences in drug absorption can be determined by delivering drug formulations directly to the upper or lower small intestine or the colon and sampling blood from the systemic circulation. In addition, collecting blood samples directly from the portal vein during parenteral as well as oral and intestinal delivery can be combined with direct portal delivery studies to tweeze apart the first pass effects of both liver and intestinal cell enzymatic degradation effects on the drug being tested. Next, delivery of various formulations under different conditions can help in the development of effective oral delivery systems. Finally, delivery of absorption enhancers at various intervals, prior to the delivery of the drug, can be used to determine the recovery time of intestinal wall integrity following dosing with a particular formulation.

IVAP dogs are fitted with three intestinal catheters, in addition to a portal vein catheter. Following induction of general anesthesia, the animal is placed in a supine position, scrubbed and prepped with betadine solution and draped under sterile conditions. A vertical midline incision is made through the skin and the abdominal cavity is entered. A 5F Heparin Coated CBAS® tubing, is inserted into the portal vein and secured with a purse string of 7-0 prolene suture. A small square of Surgicel® is placed over the insertion site and the fascia is closed over it to prevent leakage or dislodging of the tubing. The Upper Small Intestine (USI) port tubing is inserted 10 cm distal to the ligament of Treitz. The small bowel is then measured from the pyloric sphincter to the ileocecal valve and the Lower Small Intestine (LSI) port tubing is inserted one third of the way back from the ileocecal valve. The colon tubing is inserted 10 cm distal to the ileocecal valve (Colon). 7F Sylastic® tubing is used for each intestinal catheter. They have closed ends with a 1 cm slit to allow perfusion of drug into the lumen of the bowel, while minimizing back flow of bowel contents into the tubings. The tubings are secured with a silk “purse-string” suture and the modified Witzel Tunnel technique. After placement of the tubes, the bowel is secured to the abdominal wall and the proximal ends of the tubings are tunneled out of the abdominal cavity and into a subcutaneous pocket on the caudal aspect of the right chest, along with the PV catheter. The abdominal incision is closed in two layers. The Linea Alba is approximated with 0 Vicryl and the skin is closed with 2-0 Vicryl.

The animal is then turned to a left lateral recumbent position and is re-scrubbed and draped. The lateral pocket is opened and the incision extended to allow for creation of a subcutaneous pocket along the back of the animal. The port tubings are attached to their respective reservoirs. These reservoirs are secured along the spine to the tough fascia layer with 0 Prolene sutures. Each port is accessed, flushed and checked for leakage. The subcutaneous space is dowsed with Ampicillin powder and closed with 2-0 vicryl.

Post-op antibiotic treatment with 7 mg/kg Ampicillin and 2 mg/kg Gentamicin SQ BID continues for 10 days. When the animal is fully recovered (minimum 2 weeks) and all antibiotic treatments have been completed, the animals are tested as frequently as once a week. Hematocrits are monitored and animals are given an extra week rest period if the hematocrtit drops below 35%. Also, because Pentostatin is an immunosuppressant drug and because near therapeutic doses were used, blood samples were drawn weekly for White Blood Count (WBC) determination throughout the test period. Animals were rested for an extra week if their WBC dropped below 7000 WBC/ul.

Weekly port flushing is essential to keeping the catheters patent. The skin covering the ports is scrubbed with betadine, wiped clean with alcohol and allowed to dry prior to accessing the ports. A 22 g Huber needle is used to access each port without coring the septum. After withdrawal of the old heparin lock solution, the vascular port is flushed with 3 ml of sterile saline and refilled with fresh, 1000 units/ml heparin in 50% Dextrose (D50).

Once a month, the old heparin lock solution that is withdrawn from the PV port during flushing, is cultured in order to monitor for infection. When a positive culture is found, the organism is identified and its antibiotic sensitivity is determined. The antibiotic to which it is most sensitive and which is available in liquid form, is used to treat the infection. PV port infections are treated by using the highest concentration available of the liquid antibiotic solution and mixing it with 10,000 units per ml heparin solution to produce an “antibiotic hep-lock” solution containing 500 units/ml heparin. This solution is used to refill the port and catheter after flushing it clear with sterile saline. This solution is removed, flushed and replaced every 12 hours for 2 days. The port is then refilled with 1000 units/ml antibiotic heparin solution twice more at 3 day intervals. At the end of the treatment, the antibiotic solution is withdrawn and the port and catheter are flushed clean with 5 to 10 ml of sterile saline and refilled with the usual 1000 units/ml heparin lock (D50) solution. Three or 4 days later, this solution is withdrawn and cultured to insure that the infection is cured. During antibiotic treatment, the animal is taken off the test schedule. They are returned only after a 5 day withdrawal period following completion of the treatment. There did not happen to be any PV infections in the individuals on this study during the time period covered by this report.

Negative pressure is never applied to an intestinal port; the catheter is simply flushed and filled with 2 to 3 ml of 50% Dextrose solution once a week.

On the day prior to a study, each port that will be used the next day is flushed with 3 to 5 ml of sterile saline. During this procedure the needle is rotated to insure all aspects of the port body are flushed clear.

Following the last sample on the day of a study, all intestinal ports are refilled with 2 to 3 ml of D50. In cases where PV sampling was performed, the PV port would also be flushed clear with 3 to 5 ml of saline and refilled with 1 ml of 1000 units/ml heparin in D50.

The test material is prepared and administered by using the following protocol:

i) On the day prior to an intestinal infusion study, all ports would be flushed according to the above SOP, except that the port intended for use, would be flushed clear with sterile saline.

ii) On the morning of the test, each animal is weighed and placed in the sling. An IV is installed in the brachial vein for systemic blood sampling with a multiuse vacutainer needle adapter. This is flushed with heparinized saline (50 units/ml) to maintain patency between sampling. When necessary, a 20 g or 22 g right angled huber infusion set would be placed in the PV port and attached to a three-way stopcock for PV blood sampling throughout the test.

iii) Infusate was prepared as follows:

In case of IV administration, the drug is dissolved in normal sterile saline to a concentration of 1 mg/ml and is passed through a 22 um sterilizing filter prior to administration to the brachial vein. In the case of all other infusions, the drug is dissolved in either sterile water for injection (SWFI) or Phosphate Buffer to a concentration of 1 mg/ml and drawn up into dosing syringes. All animals were dosed at a rate of 0.2 mg/kg for each permutation.

iv) Dosage is administered as follows:

In case of IV administration, a second IV catheter is installed and used to dose the animal. The entire volume is delivered within 45 seconds. In the case of oral dosages, the dose is delivered to the back of the mouth with an appropriate size syringe and is followed with 5 ml of tap water to assist swallowing and clearance of the dose from the mouth. For all other tests, the dosage is infused into the appropriate intestinal port and flushed with 2-3 ml of sterile saline. The deadspace volume of the ports and tubings vary slightly between individuals, but are generally less than 0.5ml total. Hence, the four to six fold volume flush is accepted as sufficient to insure complete clearing of the dosage from the port and tubing.

The blood samples were collected by using the following protocol:

i) Regular sampling from systemic and portal veins occurred at 5 and 15 minutes as well as 1, 2 and 6 hours following dosing. An additional systemic only sample was taken at 12 hours post-dose.

ii) Following the 6 hour blood sample, all catheters and infusion sets are removed and the animal is returned to his run an fed. In cases where PV sampling is being performed the infusion set, port and catheter are flushed and filled with heplock solution.

iii) A few minutes prior to the 12 hour sample, the animals are returned to the slings and prepared for the final sample. A 20 g vacutainer needle is used to collect the final systemic sample.

iv) Following the final blood sample, the intestinal ports are flushed and refilled with 2 to 3 ml of 50% Dextrose solution in order to prevent the back flow of ingesta and bile salts.

The blood samples collected were processed by using the following protocol:

All blood samples are kept on ice and spun at 1000×g in a refrigerated centrifuge at 2 to 4° C. within 30 minutes of being drawn. The plasma is then decanted into labeled microcentrifuge tubes and frozen at −24° C. Upon completion of study day, the samples are transferred to a −70 deg C. freezer while awaiting shipment to the analytical lab.

2) Bioavailability and Absorption of Pentostatin

a) Effect of Buffered Formulation on Bioavailability

The bioavailability of pentostatin was calculated as the ratio of the extravascular and intravenous AUC calculated with peripheral blood samples. The maximum bioavailability was obtained after infusion of pentostatin in the jejunal port (F=0.539) and the lowest in the colon (F-0. 119) that also presented the highest variability (CV=159 %).

Administration of Pentostatin in a 100 mM pH=7 PO₄ buffer solution has a significant effect in the systemic exposure measured as AUC from time 0 to infinite. In this case, jejunum also presented the highest bioavailability (F=0.879) but oral administration presented the lowest (F=0.143). However, the biggest effect (Table 1, column 3) of the buffered solution was achieved in ileum followed by colon and a negative effect was observed after oral administration.

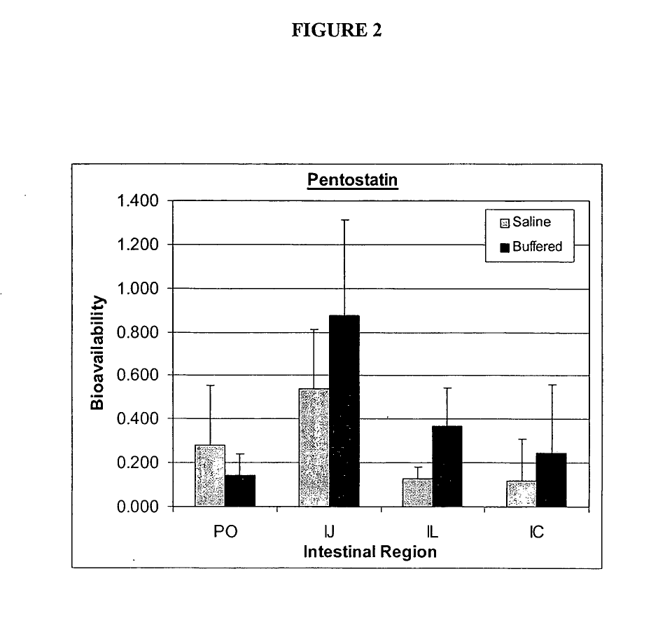

Table 1 and FIG. 2 show a summary generated with the average bioavailability values obtained from systemic blood samples. The complete individual data are shown in Table 2.

TABLE 1
Effect of buffer in systemic bioavailability

| | | | Ratio |
|---|---|---|---|
| Route of | Treatment 1 - | Treatment 2 - | Treat₂/ |
| Administration | Saline | Buffered | Treat₁ |
| Oral | 0.280 ± 0.272 (97) | 0.143 ± 0.098 (69) | 0.51 |
| Intrajejunal | 0.539 ± 0.273 (51) | 0.879 ± 0.434 (49) | 1.63 |
| Intraileum | 0.127 ± 0.054 (43) | 0.367 ± 0.176 (48) | 2.88 |
| Intracolon | 0.119 ± 0.188 (159) | 0.244 ± 0.315 (129) | 2.05 |

Coefficient of variation in parenthesis (%)

The systemic exposure or AUC values from time 0 to infinite is summarized in Table 2.

TABLE 2
Effect of buffer in systemic AUC_{0-inf}
after extravascular administration

| Route of | Mean systemic AUC | Mean systemic AUC |
|---|---|---|
| Administration | Treatment 1 - Saline | Treatment 2 - Buffered |
| Oral | 20,408 ± 21,426 (105) | 10,425 ± 6,652 (64) |
| Intrajejunal | 39,029 ± 14,099 (36) | 64,609 ± 27,409 (42) |
| Intraileum | 10,641 ± 4,324 (41) | 30,013 ± 11,930 (40) |
| Intracolon | 13,274 ± 17.891 (135) | 27,356 ± 27,390 (100) |

Coefficient of variation in parenthesis (%)
AUC_{0-inf} units: ng × min/ml

The data in Table 1 and Table 2 as well as FIG. 2 indicate the following:

i) The effect of the buffered solution is not uniform along the GI. The highest effect is found in the ileum where systemic bioavailability in treatment 2 was found to be 2.88 times the value found for treatment 1. Colon and jejunum regions also showed increases (2.05 and 1.63 times) in reference to the bioavailability found for treatment 1;

ii) Oral administration of-pentostatin in buffer vehicle caused a 50% decrease in the systemic pentostatin bioavailability; and

iii) Administration of pentostatin in the buffered solution seems to decrease the variability.

The individual bioavailability data are listed in Table 3.

TABLE 3
Individual bioavailability data depending
on route of administration.

| | | Route | | | |
|---|---|---|---|---|---|
| Animal ID | Treatment | PO | IJ | IL | IC |
| RS-65 | Saline | 0.219 | 0.82 | N/A | N/A |
| | Buffered | 0.230 | 1.28 | N/A | N/A |
| | % change | 5% | 56% | N/A | N/A |
| RS-66 | Saline | 0.042 | 0.28 | 0.19 | 0.01 |
| | Buffered | 0.037 | 0.42 | 0.31 | 0.10 |
| | % change | −12% | 50% | 64% | 636% |
| RS-67 | Saline | N/A | 0.51 | 0.10 | 0.01 |
| | Buffered | N/A | 0.93 | 0.23 | 0.03 |
| | % change | N/A | 82% | 121% | 320% |
| RS-68* | Saline | 0.577 | N/A | N/A | 0.34 |
| | Buffered | 0.160 | N/A | N/A | 0.61 |
| | % change | −72% | N/A | N/A | 80% |
| RS-69* | Saline | N/A | N/A | 0.09 | N/A |
| | Buffered | N/A | N/A | 0.57 | N/A |
| | % change | N/A | N/A | 529% | N/A |
| AVERAGE | Saline | 0.280 | 0.538 | 0.127 | 0.119 |
| | Buffered | 0.143 | 0.879 | 0.367 | 0.244 |
| | % change | −26% | 63% | 238% | 345% |
| STANDARD | Saline | 0.272 | 0.273 | 0.054 | 0.188 |
| DEVIATION | Buffered | 0.098 | 0.434 | 0.176 | 0.315 |
| | % change | 15% | 90% | 39% | 29% |

*= F calculated in reference to the IV average AUC obtained with animals RS-65, RS-66 and RS-67

It is noted that administration of pentostatin with the buffered solution has a negative effect on systemic bioavailability.

b) Preferential Intestinal Absorption Region

The systemic exposure values indicate that after extravascular administration the higher AUC is obtained after intra jejunal administration. Table 4 shows the relative bioavailability taking as a reference the results from the oral administration in each treatment.

TABLE 4
Relative bioavailability (%) in reference to oral

| Route of | Bioavailability | Bioavailability |
|---|---|---|
| Administration | Treatment 1 - Saline | Treatment 2 - Buffered |
| Oral | 100 | 100 |
| Intrajejunal | 191 | 620 |
| Intraileum | 52 | 288 |
| Intracolon | 65 | 262 |

In addition to systemic blood samples, portal vein samples were also taken. Measuring drug concentration in portal vein can give a better indication of preferential intestinal absorption since it avoids potential loses due to hepatic first pass elimination (but not intestinal-wall related loss). The mean AUC values obtained in portal vein are shown in Table 5.

TABLE 5
Effect of buffer in mean portal vein AUC_{0-inf} after extravascular administration

| Route of | | | |
|---|---|---|---|
| Administration | Treatment 1-Saline | Treatment 2-Buffered | AUC₂/AUC₁ |
| Oral | 8,277 ± 7,607 (92) | 12,416 ± 14,105 (114) | 1.50 |
| Intrajejunal | 30,254 ± 12,501 (41) | 51,280 ± 16,769 (33) | 1.69 |
| Intraileum | 13,905 ± 3,134 (23) | 18,367 ± 3,004 (16) | 1.32 |
| Intracolon | 2,011 ± 1,964 (98) | 5,114 ± 2,582 (50) | 2.54 |

Coefficient of variation in parenthesis (%)
AUC_{0-inf} units: ng x min/ml

AUC values in the portal vein also increased after administration in the buffered solution for all the routes tested. Similarly, variability was also decreased except in oral administration. If the data in Table 5 are transformed to evaluate the relative bioavailability we obtain the following results (Table 6):

TABLE 6
Relative bioavailability calculated in portal vein

| Route of | | |
|---|---|---|
| Administration | Treatment 1 - Saline | Treatment 2 - Buffered |
| Oral | 100 | 100 |
| Intrajejunal | 365 | 413 |
| Intraileum | 167 | 147 |
| Intracolon | 24 | 41 |

The data presented in Table 5 and 6 clearly confirm the preferential absorption of pentostatin in the jejunum segment of the intestine. The pattern is also maintained in both treatments. In addition, we can also notice that the pattern differ from that defined with systemic blood samples (Table 4). The portal vein relative bioavailability calculated for ileum and colon are very different while their systemic relative bioavailability is similar. This may reflect the anatomical fact that certain veins that irrigate the colon segment do not drain into the portal vein system and bypass detection in the blood samples from the portal vein.

3) Pharmacokinetic Profiles

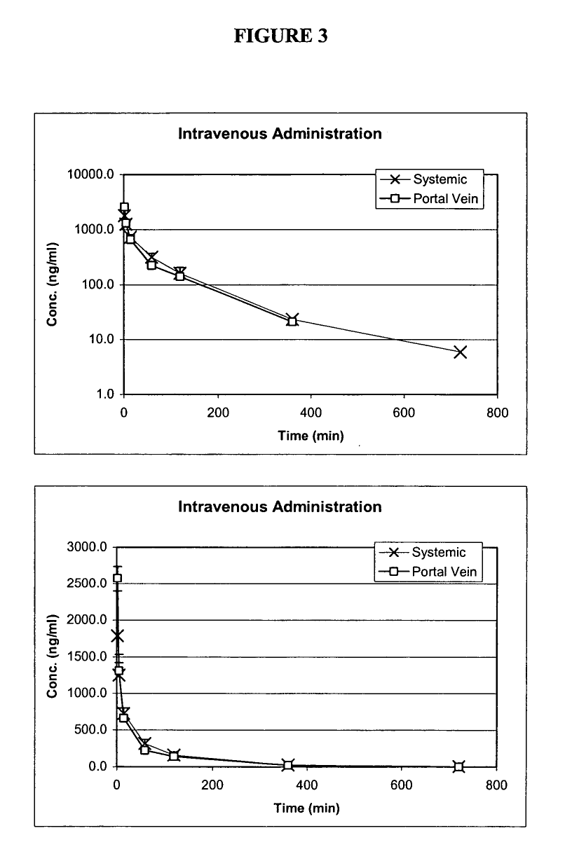

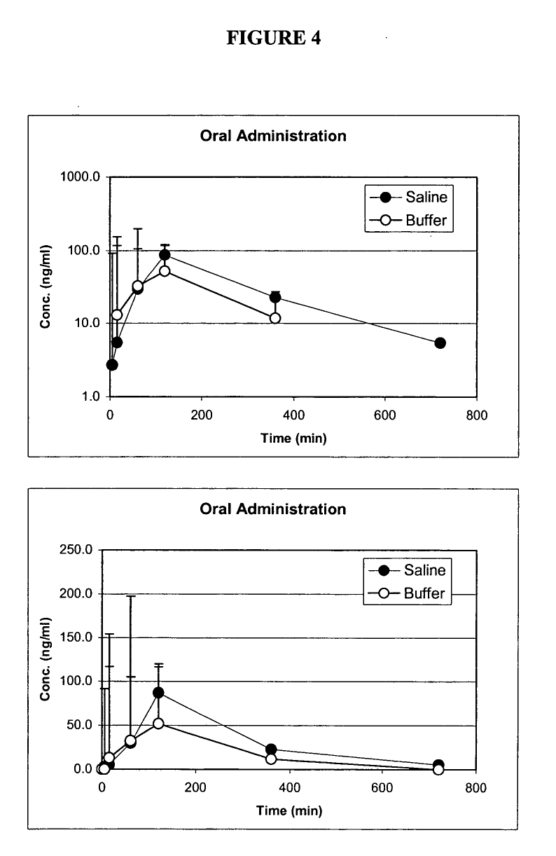

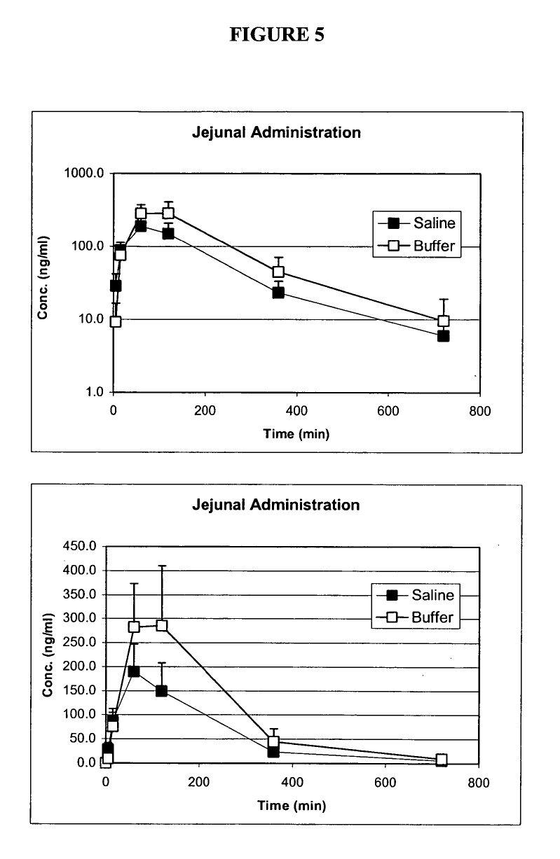

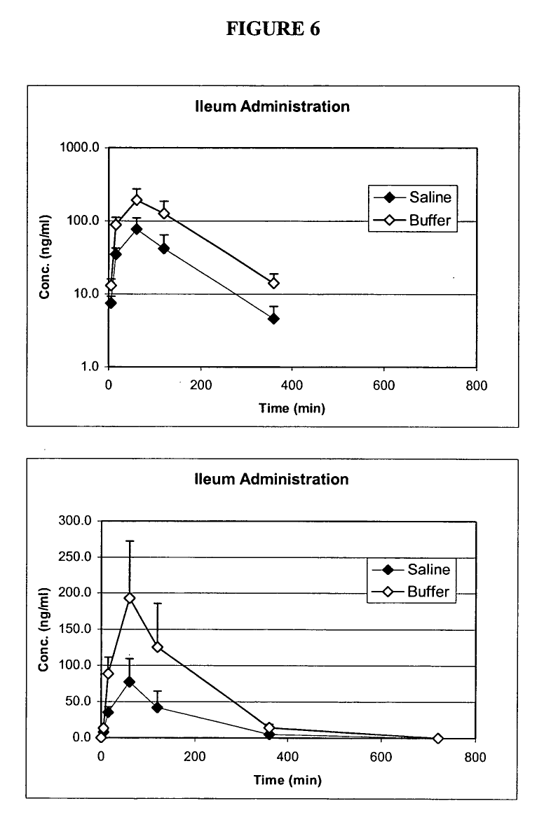

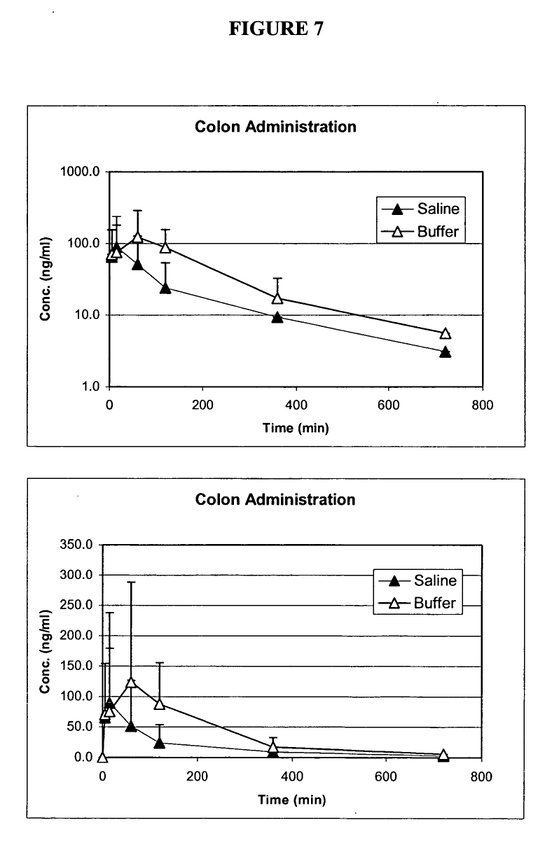

The pharmacokinetic profiles of pentostatin via intravenous (FIG. 3), oral (FIG. 4), jejunal (FIG. 5), ileum (FIG. 6), and intracolon (FIG. 7) administration are presented in both log and natural scales and represent the average values for each time point.

a) Pharmacokinetics of Decitabine in IVAP Rabbit Model

To determine whether decitabine can be preferably absorbed in a specific region(s) of the GI tract with high bioavailability, pharmacokinetics studies of decitabine were designed and performed as summarized in the following.

Decitabine was administered intravenously to IVAP rabbits (n=3) at three doses: 0.75, 1.5 and 2.5 mg/kg. It was also administered at 2.5mg/kg orally and through previously implanted intestinal and vascular access ports in the portal vein (PV), upper small intestine (USI), lower small intestine (LSI) and colon (IC). All routes received the same simple formulation of raw compound dissolved in cold saline. Care was taken to insure doses were delivered immediately (less than 15 minutes) after dissolving the powder and that the liquid formulation was kept on ice up to the time of administration. Blood samples were taken simultaneously from the median auricular artery and through a port in the portal vein at predetermined time points. Plasma was separated and kept frozen at −70° C. until shipped to Xenobiotic Laboratories for analysis.

Pharmacokinetic parameters were calculated using non-compartmental models with the Winnonlin v3.1 program.

i) Dose Linearity

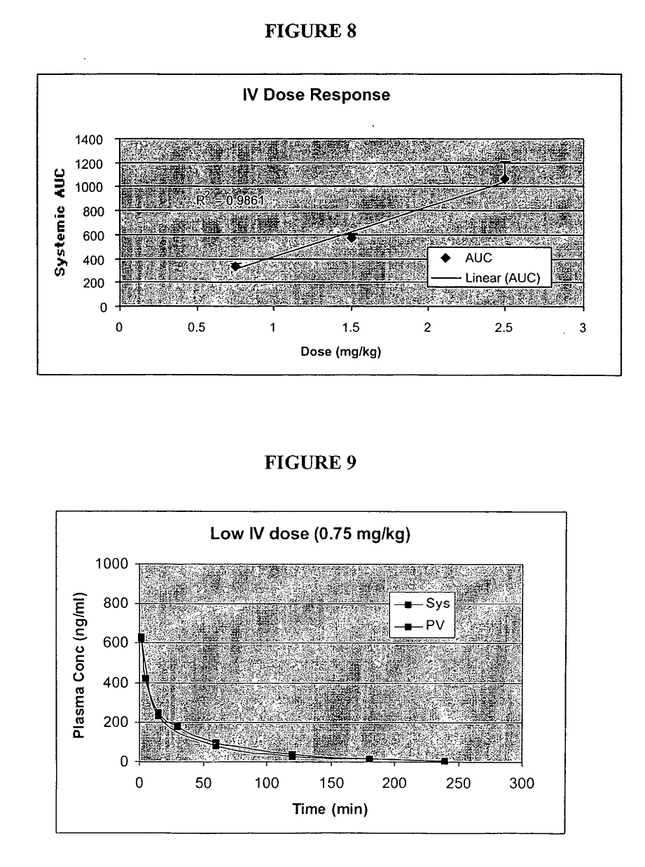

Dose Linearity was observed for the three IV doses tested. Individual AUC data as well as linearity chart are presented in Table 7 and plotted in FIG. 8.

TABLE 7
IV Area Under the Curve (AUC) data

| | Dose IV | | |
|---|---|---|---|
| | 0.75 mg/kg | 1.5 mg/kg | 2.5 mg/kg |
| | 322.63 | 557.43 | 1166.97 |
| | 327.37 | 552.87 | 896.21 |
| | 353.53 | 612.34 | 1128.11 |
| | 354.68 | | |
| Mean | 339.55 | 574.21 | 1063.77 |
| Std Dev | 16.92 | 33.10 | 146.40 |

AUC_{0-inf} units: hr × ng/ml

ii) Bioavailability

Bioavailability of Decitabine was found to be site dependent and individual data are listed in Table 8. The highest bioavailability was achieved after administration in the USI (F=73.9%) and was very similar to direct portal administration (67.9%). Administration via the USI port yielded the highest Cmax and AUC of all extravascular routes. Oral delivery produced the lowest mean bioavailability (35.5%) and colonic delivery produced the most variable results (0.2 to 68.3%). In the case of the colonic dosing, RU-41's catheter had become dislodged with no symptoms to indicate there was a problem. Hence, the dose was inadvertently delivered intraperitoneally. The dose was nearly completely absorbed, giving a pK profile similar to an IV dose. Despite the low number of successful colon infusions, it is clear that colonic delivery results in extremely high variability. If the sponsor is interested, several additional animals would need to be prepared and studied in order to better understand this phenomenon.

TABLE 8
Individual systemic Bioavailability:
portal and GI administration

| | Route | | | | |
|---|---|---|---|---|---|
| Animal ID | PV | PO | USI | LSI | IC |
| RU-35 | | 39.12% | 72.64% | | |
| RU-41 | | 28.57% | | | IP dose |
| RU-44 | | 38.84% | | | 0.22% |
| RU-45 | | | | | 68.29% |
| RU-46 | | | 64.84% | | |
| RU-48 | 79.63% | | | 55.05% | |
| RU-49 | 58.11% | | | 44.48% | |
| RU-50 | 66.02% | | | 51.78% | |
| RU-52 | | | 84.33% | | |
| Mean | 67.92% | 35.51% | 73.94% | 50.44% | 34.26% |
| Std Dev | 10.88% | 6.01% | 9.81% | 5.41% | 48.13% |

iii) First Pass Effect

In addition to systemic blood samples, portal vein samples were also taken. Measuring drug concentration in the portal vein gives additional information on absorption rate and intestinal metabolism of the drug. The mean AUC values obtained in the portal vein as well as systemic samples are shown in Table 9. The higher variability in the PV samples is partly due to difficulty in sampling. In some cases sampling was very difficult and sporadic. In the first of the USI dosings, no PV sampling was possible. With only 2 sets of data, from the animals with the lowest and highest systemic AUC's, the low “n” is responsible for the higher variability in this case.

When the same dose is delivered directly into the portal vein, the 68% bioavailability indicate that the hepatic first pass effect results in 32% loss ((AUCIV−AUCPV/AUCIV)×100). Also, following IV dosing, systemic and portal vein sample concentrations are nearly identical throughout the sampling period, which indicates there is no significant intestinal metabolism of Decitabine in the rabbit.

The USI bioavailability (74%) is similar to that when delivered into the PV, which indicates complete absorption of Decitabine through the Upper Small Intestine. The lower bioavailability using other extravascular routes demonstrates reduced and variable absorption from the stomach, Lower Small Intestine and Colon.

TABLE 9
Mean AUC_{0-inf} after extravascular administration

| Route of | AUC in Systemic | |
|---|---|---|
| Administration | Samples | AUC in Portal Vein |
| Oral | 402 ± 68.0 (16.9%) | 397 ± 53.7 (13.5%) |
| USI | 837 ± 111 (13.3%) | 833 ± 275 (33.0%) |
| LSI | 571 ± 61.3 (10.7%) | 586 ± 42.1 (7.18%) |
| IC (only two | 2.51 and 773 (140%) | 2.98 and 830 (140%) |
| successful doses) | | |

Coefficient of variation in parenthesis (%)
AUC_{0-inf} units: hr × ng/ml

iv) Elimination Half-Life and Mean Residence Time

Elimination half-life and Mean Residence Time were similar regardless of dose or route of administration. The most variability in these parameters was recorded following oral administration. Mean data with standard deviations are presented in Table 10.

TABLE 10
Terminal half-life and Mean Residence Time data

| Dose + Route of | Mean half-life | Mean Residence |
|---|---|---|
| Administr. | (hours) | Time (hr) |
| 0.75 mg/kg IV | 0.73 ± 0.03 | 0.84 ± 0.03 |
| 1.5 mg/kg IV | 0.75 ± 0.04 | 0.83 ± 0.03 |
| 2.5 mg/kg IV | 0.59 ± 0.02 | 0.83 ± 0.15 |
| 2.5 mg/kg PV | 0.77 ± 0.07 | 0.80 ± 0.06 |
| 2.5 mg/kg Oral | 0.69 ± 0.26 | 1.21 ± 0.40 |
| 2.5 mg/kg USI | 0.79 ± 0.09 | 0.92 ± 0.08 |
| 2.5 mg/kg LSI | 0.74 ± 0.06 | 0.92 ± 0.09 |
| 2.5 mg/kg IC | 0.68 ± 0.01 | 1.07 ± 0.02 |
| Overall mean | 0.72 ± 0.07 | 0.93 ± 0.11 |

v) Maximum Concentration and Time of Maximum

The highest plasma concentration (Cₘₐₓ) was achieved after USI administration. Shorter Tₘₐₓ correlates with higher Cₘₐₓ and AUC, indicating that rapid absorption is key to higher bioavailability (Table 11).

TABLE 11
Mean Cₘₐₓ and Tₘₐₓ data for extravascular administration

| Route of | Mean Cₘₐₓ | Mean Tₘₐₓ |
|---|---|---|
| Administration | (ng/ml ± Std Dev) | (min) |
| Oral | 335 ± 134 | 0.42 ± 0.14 |
| USI | 1218 ± 280 | 0.083 ± 0.0 |
| LSI | 736 ± 95.6 | 0.14 ± 0.096 |
| IC | 2.0 and 611 | 0.25 ± 0.0 |

vi) Clearance and Volume of Distribution

Decitabine clearance (Cₗ) and Volume (V_{z}) were calculated for each of the systemic profiles using non-compartmental models with the Winnonlin v3.1 program. The results are shown in Table 12 as normalized data for body weight. Clearance and Volume of distribution values were very consistent throughout all IV doses and GI routes of administration, but were significantly higher following direct portal vein infusion.

TABLE 12
Mean Cₘₐₓ and Tₘₐₓ data
for extravascular administration

| Route of | Clearance | Volume |
|---|---|---|
| Administration | (L/hr/kg) | (L/kg) |
| PV | 3.31 | 3.63 |
| Oral | 2.26 | 2.29 |
| USI | 2.23 | 2.51 |
| LSI | 2.23 | 2.37 |
| IC | 2.21 | 2.16 |
| IV (overall) | 2.40 ± 0.20 | 2.41 ± 0.41 |

Individual Pharmacokinetic Data

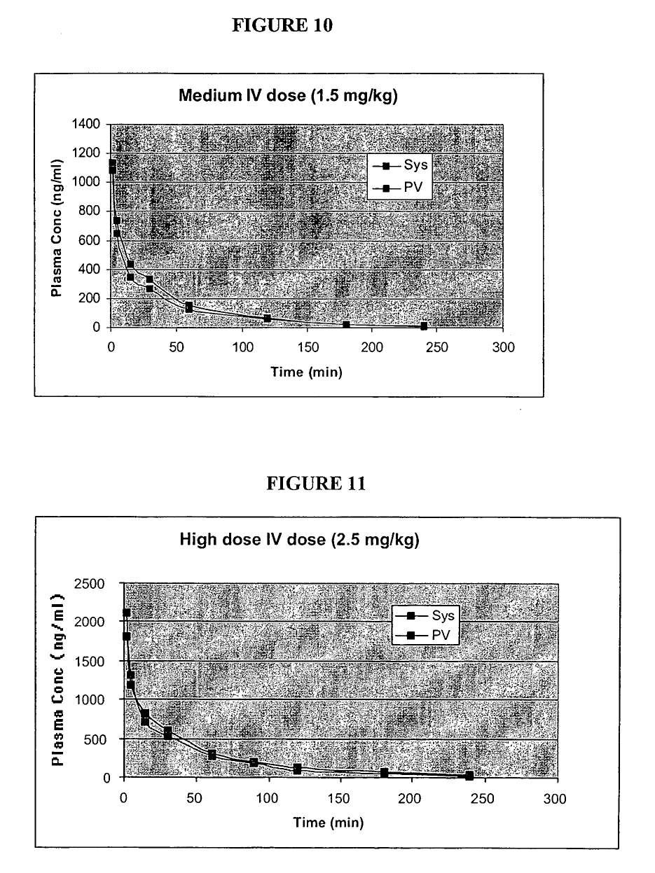

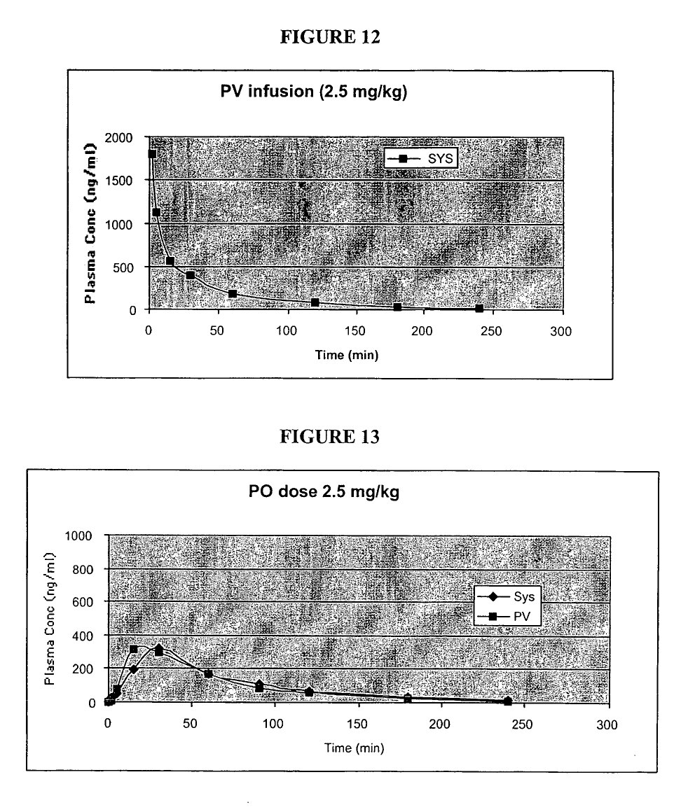

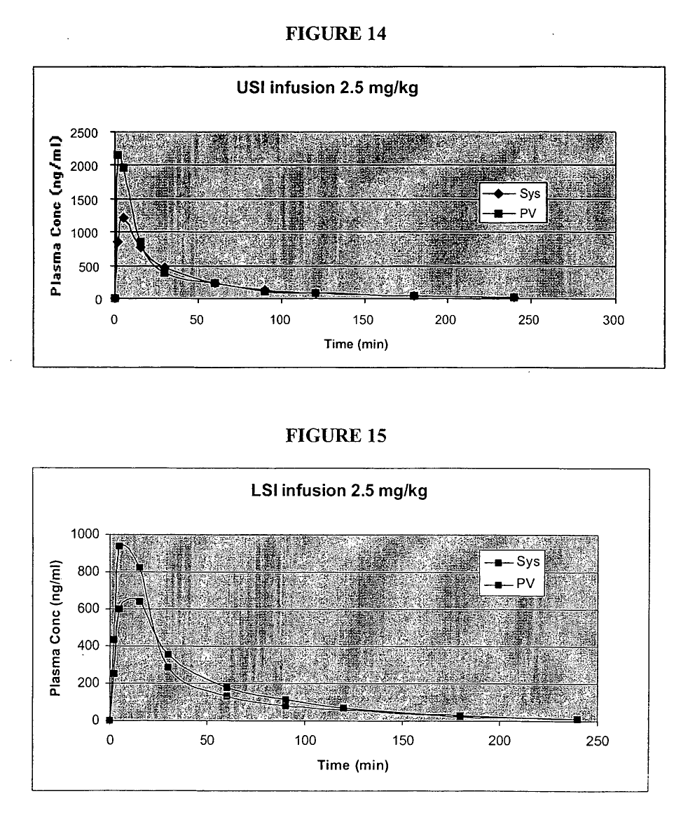

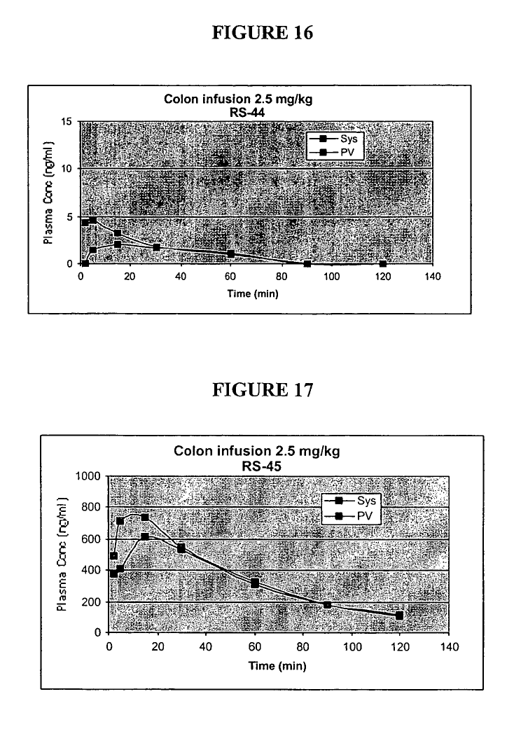

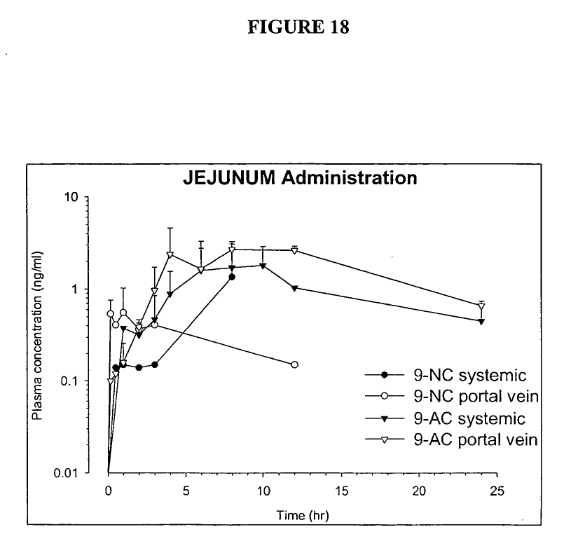

The concentration versus time data for intravenous administration (Tables 13, 14, and FIGS. 9, 10, and 11), portal venous administration (Table 16 and FIG. 12), peronral administration (Table 17 and FIG. 13), local administration in upper small intestine region (Table 18 and FIG. 14), local administration in lower small intestine region (Table 19 and FIG. 15), and local administration in colon (Table 20 and FIGS. 16 and 17) are shown below.

i) Intravenous Administration

TABLE 13
Individual time point data after IV administration (0.75 mg/kg)

| | Animal ID | | | | | | | | | |
|---|---|---|---|---|---|---|---|---|---|---|
| | RU-48 | | RU-44 | | RU-45 | | | | | |
| | Date + Weight | | | | | | | | | |
| | Aug. 28, | | Jul. 10, | | Jul. 16, | | | | | |
| | 2002 | | 2002 | | 2002 | | IV 0.75 mg/kg | | | |
| | 3.7 kg | | 4.1 kg | | 4.4 kg | | SYS | | PV | |
| Time (min) | SYS | PV | SYS | PV | SYS | PV | Mean | SD | Mean | SD |
| 0 | 0 | 0 | 0 | 0 | 0 | 0 | 0.0 | 0.0 | 0.0 | 0.0 |
| 2 | 666 | 581 | 615 | 621 | 603 | 669 | 628 | 33 | 624 | 44 |
| 5 | 432 | — | 415 | — | 419 | — | 422 | 9 | — | — |
| 15 | 257 | 233 | 233 | 220 | 250 | 233 | 247 | 12 | 229 | 8 |
| 30 | 177 | — | — | — | — | — | 177 | — | — | — |
| 60 | 102 | 95.1 | 89.1 | 65.8 | 102 | 89.1 | 98 | 7 | 83 | 15 |
| 120 | 40.6 | — | 35.2 | 21.1 | 41.0 | 34.0 | 39 | 3 | 28 | — |
| 180 | 15 | 13.9 | 13.9 | — | 15.7 | — | 15 | 1 | 14 | — |
| 240 | 5.9 | — | 6.21 | 4.87 | 6.31 | 5.76 | 6 | 0 | 5 | — |

TABLE 14
Individual time point data after IV administration (1.5 mg/kg)

| | Animal ID | | | | | | | | | |
|---|---|---|---|---|---|---|---|---|---|---|
| | RU-50 | | RU-44 | | RU-35 | | | | | |
| | Date + Weight | | | | | | | | | |
| | Oct. 14, | | Jun. 25, | | Apr. 3, | | | | | |
| | 2002 | | 2002 | | 2002 | | IV 1.5 mg/kg | | | |
| | 3.8 kg | | 4.0 kg | | 4.43 kg | | SYS | | PV | |
| Time (min) | SYS | PV | SYS | PV | SYS | PV | Mean | SD | Mean | SD |
| 0 | 0 | ND | 0 | 0 | 0 | 0 | 0 | 0 | 0 | 0 |
| 2 | 1240 | 1297 | 919 | 1132 | — | 985 | 1080 | — | 1138 | 156 |
| 5 | 819 | — | 676 | — | 711 | 652 | 735 | 75 | 652 | — |
| 15 | 443 | 352 | 404 | 301 | 453 | 397 | 433 | 26 | 350 | 48 |
| 30 | 355 | — | — | — | 304 | 266 | 330 | — | 266 | — |
| 60 | 149 | 124 | 148 | 119 | 171 | 141 | 156 | 13 | 128 | 44 |
| 120 | 62.6 | 60 | 64.9 | 57.2 | 60.3 | 58.8 | 63 | 2 | 59 | 22 |
| 180 | 23.7 | — | 25.3 | 22.1 | 23 | 21.1 | 24 | 1 | 22 | — |
| 240 | 10.2 | 13.6 | 10.8 | — | 9.42 | 9.67 | 10 | 1 | 12 | — |

TABLE 15
Individual data and pharmacokinetic parameters
after IV administration (2.5 mg/kg)

| | Animal ID | | | | | | | | | |
|---|---|---|---|---|---|---|---|---|---|---|
| | RU-41 | | RU-45 | | RU-49 | | | | | |
| | Date + Weight | | | | | | | | | |
| | Jun. 3, | | Jun. 25, | | Aug. 28, | | | | | |
| | 2002 | | 2002 | | 2002 | | IV 2.5 mg/kg | | | |
| | 4.9 kg | | 4.2 kg | | 3.9 kg | | SYS | | PV | |
| Time (min) | SYS | PV | SYS | PV | SYS | PV | Mean | SD | Mean | SD |
| 0 | 0 | 0 | 0 | 0 | 0 | — | 0 | | 0 | — |
| 2 | 1947 | 2579 | 1488 | 1666 | 1963 | — | 1799 | 270 | 2123 | — |
| 5 | 1288 | 1322 | 1131 | — | 1152 | — | 1190 | 85 | 1322 | — |
| 15 | 859 | 759 | 725 | 669 | 888 | — | 824 | 87 | 714 | — |
| 30 | 656 | 536 | — | — | 565 | — | 611 | 64 | 536 | — |
| 60 | 334 | 281 | 269 | 264 | 339 | — | 314 | 39 | 273 | — |
| 90 | 202 | 185 | — | — | — | — | 202 | — | 185 | — |
| 120 | 140 | 102 | 75.6 | 80 | 124 | — | 134 | 34 | 91 | — |
| 180 | 26.6 | 51.5 | 24.2 | — | 29.5 | — | 65 | 3 | 51.5 | — |
| 240 | 59.9 | 23.2 | 10.5 | 8.86 | 26.1 | — | 32 | 25 | 16 | — |

ii) Portal Venous Administration

TABLE 16
Individual time point data after PV administration (2.5 mg/kg)

| Animal ID | | | |
|---|---|---|---|
| RU-48 | RU-49 | RU-50 | PV |
| Oct. 2, 2002 Weight | | | 2.5 mg/kg |
| 3.9 kg | 3.7 kg | 3.7 kg | SYS |

| Time (min) | SYS | SYS | SYS | Mean | SD |
|---|---|---|---|---|---|
| 0 | 0 | 0 | 0 | 0 | 0 |
| 2 | 2076 | 1592 | 1731 | 1800 | 249 |
| 5 | 1241 | 1029 | 1092 | 1121 | 109 |
| 15 | 623 | 485 | 598 | 569 | 74 |
| 30 | 425 | 302 | 356 | 391 | 62 |
| 60 | 228 | 169 | 168 | 188 | 34 |
| 120 | 93.7 | 64.5 | 85.5 | 81 | 15 |
| 180 | 47 | 26 | 27.7 | 34 | 12 |
| 240 | 18 | 8.55 | 12.5 | 13 | 5 |

iii) Oral Administration

TABLE 17
Individual time point data after Oral administration (2.5 mg/kg)

| | Animal ID | | | | | | | | | |
|---|---|---|---|---|---|---|---|---|---|---|
| | RU-35 | | RU-41 | | RU-44 | | | | | |
| | Date + Weight | | | | | | | | | |
| | Jun. 25, | | Jun. 12, | | Jun. 12, | | | | | |
| | 2002 | | 2002 | | 2002 | | PO 2.5 mg/kg | | | |
| | 4.6 kg | | 4.9 kg | | 3.9 kg | | SYS | | PV | |
| Time (min) | SYS | PV | SYS | PV | SYS | PV | Mean | SD | Mean | SD |
| 0 | 0 | 0 | 0 | 0 | 0 | 0 | 0 | 0 | 0 | 0 |
| 2 | 83.8 | 14.2 | 0 | 3.68 | 2.27 | 0 | 29 | 48 | 6 | 7 |
| 5 | 135 | 172 | 11.8 | 58.7 | 5.59 | 5.14 | 51 | 73 | 79 | 85 |
| 15 | 267 | 490 | 227 | 357 | 83.2 | 99.7 | 192 | 97 | 316 | 198 |
| 30 | 485 | 381 | 188 | 220 | 294 | 287 | 322 | 151 | 296 | 81 |
| 60 | 129 | 176 | 168 | 185 | 212 | 155 | 170 | 42 | 172 | 15 |
| 90 | — | 86.7 | 81.4 | 71 | 146 | 84.4 | 114 | 46 | 81 | 8 |
| 120 | — | 52.1 | 55.6 | 49.6 | 75.8 | 77.9 | 66 | 14 | 60 | 16 |
| 180 | — | 17.6 | 22.9 | — | 40.9 | 41.4 | 32 | 13 | 30 | 17 |
| 240 | — | 5.9 | 9.75 | 6.25 | 19.9 | 15.9 | 15 | 7 | 9 | 6 |

iv) Upper Small Intestine Administration

TABLE 18
Individual time point data after USI administration (2.5 mg/kg)

| | Animal ID | | | | | | | | | |
|---|---|---|---|---|---|---|---|---|---|---|
| | RU-35 | | RU-46 | | RU-52 | | | | | |
| | Date + Weight | | | | | | | | | |
| | Jun. 3, | | Jul. 16, | | Oct. 14, | | | | | |
| | 2002 | | 2002 | | 2002 | | USI 2.5 mg/kg | | | |
| | 4.5 kg | | 3.9 kg | | 4.9 kg | | SYS | | PV | |
| Time (min) | SYS | PV | SYS | PV | SYS | PV | Mean | SD | Mean | SD |
| 0 | 0 | 0 | 0 | 0 | 0 | 0 | 0 | 0 | 0 | 0 |
| 2 | 821 | — | 812 | 1092 | 949 | 3217 | 861 | 77 | 2155 | 1503 |
| 5 | 1167 | — | 966 | 1389 | 1520 | 2527 | 1218 | 280 | 1958 | 805 |
| 15 | 778 | 833 | 684 | 874 | 835 | 884 | 766 | 76 | 864 | 27 |
| 30 | 445 | 243 | 483 | 508 | 451 | 428 | 460 | 20 | 393 | 136 |
| 60 | 243 | 228 | 217 | 208 | 266 | 256 | 242 | 25 | 231 | 24 |
| 90 | 148 | 142 | 128 | 86 | — | — | 138 | 14 | 114 | 40 |
| 120 | 83.1 | 92.6 | 74.5 | — | 103 | — | 87 | 15 | 93 | — |
| 180 | 41.1 | 37.6 | 27 | — | — | 54.6 | 34 | 10 | 46 | 12 |
| 240 | 20.4 | 15.5 | 10.9 | — | 25.5 | — | 19 | 7 | 16 | — |

(v) Lower Small Intestine Administration

TABLE 19
Individual time point data after LSI administration (2.5 mg/kg)

| | Animal ID | | | | | | | | | |
|---|---|---|---|---|---|---|---|---|---|---|
| | RU-48 | | RU-49 | | RU-50 | | | | | |
| | Date + Weight | | | | | | | | | |
| | Aug. 8, | | Aug. 19, | | Aug. 28, | | | | | |
| | 2002 | | 2002 | | 2002 | | LSI 2.5 mg/kg | | | |
| | 3.7 kg | | 3.9 kg | | 3.5 kg | | SYS | | PV | |
| Time (min) | SYS | PV | SYS | PV | SYS | PV | Mean | SD | Mean | SD |
| 0 | 0 | 0 | 0 | 0 | 0 | 0 | 0 | 0 | 0 | 0 |
| 2 | 401 | 377 | 74 | 146 | 288 | 788 | 254 | 166 | 437 | 325 |
| 5 | 829 | 952 | 223 | 532 | 742 | 1333 | 598 | 328 | 939 | 401 |
| 15 | 605 | — | 638 | 961 | 679 | 681 | 641 | 37 | 821 | 198 |
| 30 | 359 | — | 337 | 254 | 362 | 319 | 353 | 14 | 287 | 46 |
| 60 | 195 | 134 | 165 | 114 | 179 | 143 | 180 | 15 | 130 | 15 |
| 90 | 112 | — | — | 78.3 | — | 84.8 | 112 | — | 82 | 5 |
| 120 | 71.1 | — | 67.4 | — | 61.1 | — | 67 | 5 | — | — |
| 180 | 28.1 | 21 | — | 21.4 | — | 19.2 | 28 | — | 21 | 1 |
| 240 | 11 | 9.34 | 11.4 | — | 8.95 | 8 | 10 | 1 | 9 | 1 |

vi) IntraColon Administration

TABLE 20
Individual time point data after IC administration (2.5 mg/kg)

| Animal ID | |
|---|---|
| RU-44 | RU-45 |
| Date + Weight | |
| Jun. 3, 2002 3.8 kg | Jul. 10, 2002 4.3 kg |

| Time (min) | SYS | PV | SYS | PV |
|---|---|---|---|---|
| 0 | 0 | 0 | 0 | 0 |
| 2 | 0 | 4.33 | 374 | 495 |
| 5 | 1.51 | 4.62 | 409 | 716 |
| 15 | 2 | 3.23 | 611 | 739 |
| 30 | 1.73 | 1.83 | 531 | 548 |
| 60 | 1.04 | 1.02 | 312 | 325 |
| 90 | 0 | 0 | 180 | 182 |
| 120 | 0 | 0 | 115 | 104 |

3. Pharmacokinetics of 9-Nitro-Camptothecin (9NC) in IVAP Beagle Dog Model

To Determine whether 9NC can be preferably absorbed in a specific region(s) of the GI tract with high bioavailability, pharmacokinetics studies of 9NC were designed and performed as summarized in the following.

The drug 9-Nitrocamptothecin (9-NC) was given to IVAP beagle dogs (n=3) intravenously at 0.1 and 0.2 mg/kg; infusion in the portal vein at 0.2 mg/kg; orally as suspension or capsule at 0.2 mg/kg and through intestinal ports (jejunum, ileum and colon) at 0.2 mg/kg. Blood samples were taken systemic and from the portal vein through a sampling/delivery vascular port previously implanted, then plasma was separated by centrifugation and kept frozen at −80° C. until analysis. Extracted samples were analyzed by HPLC/MS/MS and 9-NC and its metabolite 9-Aminocamptothecin (9-AC) quantified with the help of an internal standard. Model independent pharmacokinetic analysis was performed to determine the main pharmacokinetic parameters of 9-NC and 9-AC as well as 9-NC bioavailability.

After IV dosing of 9-NC proportionality between AUC (ng*hr/ml) calculated for each dose was shown (57.25±5.84 vs 118.33±21.82 for 0.1 and 0.2 mg/kg respectively). Half-life was found 0.88±0.45 vs 1.13±0.78 hrs and clearance 1.76±0.19 vs 1.73±0.78 L/hr/kg respectively. AUC values found in the portal vein were slightly lower for both doses and half-life slightly higher. Biotransformation of 9-NC and formation of 9-AC provided a metabolite/drug AUC ratio around 0.20 for both doses. The ratio was higher in the portal vein (vs systemic) suggesting certain degree of metabolism upon blood irrigation of the intestinal tissue. The metabolite reached the maximum concentration around 1 hr and showed larger half-life than the parent compound (3.51±1.87 and 6.94±2.87 hrs for 0.1 and 0.2 mg/kg doses of 9-NC respectively).

Extravascular administration of the drug yielded very low systemic 9-NC AUC values and extensive formation of 9-AC (with large systemic AUC) that resulted in low bioavailability. The systemic 9-NC exposure ranged from 0.07±0.06 after intracolon administration up to 1.65±1.15 after intraileum dosing. However, using the concentrations determined in the portal vein, the AUC was larger after PO administration (11.97±9.88) and gradually decreased along the intestine (1.48±0.29 in colon dosing). The data obtained from the portal vein seem to indicate that absorption of 9-NC occurs primarily in the upper segments of the small intestine but undergoes large metabolism upon absorption. This is suggested by the large metabolite to drug AUC ratio found in both portal vein (range 2.87 to 14.08) and systemic (ratio range 12.83 to 529.74) sampling. Administration of 9-NC in capsule form improved systemic 9-NC exposure (4.75±4.61 ng*hr/ml) giving 4.1% bioavailability but did not reduce 9-NC metabolism. In fact, more metabolite was formed probably due to lesser degradation of 9-NC in the stomach, which provided larger amount of drug available for biotransformation.

Much lesser metabolite was formed after infusion of 9-NC into the portal vein: the metabolite to drug AUC ratio was 0.76 suggesting similar biotransformation pattern to IV administration.

In conclusion, the study shows that (1) 9-NC is absorbed in the upper regions of the small intestine and (2) presents very low bioavailability due to (3) extraordinarily extensive metabolism in the intestinal wall upon absorption.

1) Materials and Methods

The test compounds (9-NC and 9-amino-camptothecin (9-AC)) and the internal standard (12-nitro-camptothecin (12-NC)) were provided by SuperGen, Inc. Regents and solvents were purchased from Sigma and Fisher, SPE columns (Spec-Plus 3 ml, C-18, catalog no: 532-03-20) from ANSYS Diagnostics (Lake Forrest, Calif.).

The samples were prepared by using the following protocol.

On analysis day, samples were thaw and extracted as follows:

Reagents: 1—Washing solution: 1% acetic acid

2—Elution solution 1:0.2% acetic acid in methanol

3—Elution solution 2:0.2% acetic acid in acetonitrile

4—Reconstitute solution:Methanol and 2% formic acid (50:50)

Procedure: 1—Aliquot 500 μl of plasma

2—Add 50 μl of internal standard

3—Add 100 μl of nanopure water

4—Vortex 30 seconds

5—Condition column:

a—0.5 ml of methanol, elute

b—0.5 ml of water, elute

6—Transfer plasma sample into the SPE column, elute

7—Wash sample

a—Add 1 ml of 1% acetic acid

b—Elute for approximate 1 min

8—Elute sample

a—Add 500 μl of 0.2% acetic acid in methanol, elute

b—Add 500 μl of 0.2% acetic acid in acetonitrile, elute.

9—Evaporate under N2 stream

10—Reconstitute with 200 μl of methanol and 2% formic acid (50/50)

11—Vortex for 1 min

12—Transfer to HPLC inserts for vial

The samples were analyzed in a Perkin Elmer liquid chromatography-tandem mass spectrometer system with a PE LC-200 Micro pump, a PE200 Autosampler and a PE Sciex API 365 mass spectrometry unit. HPLC separation was achieved with a Zorbax XDB-C18 column (4.6×150 mm, 3.5 μm). The analytical conditions are summarized in Table 21.

TABLE 21
HPLC conditions for 9-NC and 9-AC assay.

| Condition | Settings |
|---|---|
| Mobile phase | A: 0.1% Formic acid |
| | B: 0.1% Formic acid in Methanol |
| | A:B = 40:60 |
| Pump | Isocratic |
| Temperature | Room temperature |
| Flow | 0.8 ml/min |
| Injection Volume | 15 μl |

Adult male beagle dogs (between the ages of 1 and 5 years and weighed between 9 and 16 kg) were obtained from Summit Ridge Farms, Susquehanna, Pa. The IVAP dogs were prepared by using a protocol similar to the one used for pentostatin experiments described above.

On the day prior to a study, each intestinal port that will be used the next day is flushed with 3 to 5 ml of sterile saline. During this procedure the needle is rotated to insure all aspects of the port body are flushed clear.

On the morning of the test, each animal is weighed and placed in the sling. An IV catheter is installed in the brachial vein for systemic blood sampling with a multiuse vacutainer needle adapter. This is flushed with heparinized saline (50 units/ml) to maintain potency between sampling. When necessary, a 20 g or 22 g right-angled Huber infusion set would be placed in the PV port and attached to a three-way stopcock for PV blood sampling.

The preparation of the dose depends on the route of study. In case of IV and PV administration, the drug was dissolved in dimethylacetamide (DMA) at 5 mg/ml and 0.5 ml of the DMA concentrate filtered through a 22 μm-sterilizing filter into 4.5 ml of diluent (51% PEG-400 49% 0.001 M H₃PO₄) prior to administration to the brachial or portal vein. In the case of all other infusions, as well as oral administration, the drug was dissolved in vegetable oil to a concentration of 1 mg/ml and drawn up into dosing syringes. Animals were dosed IV at 0.1 and 0.2 mg/kg and 0.2 mg/kg for all the other routes of administration tested.

In intravascular studies, the dose is administered via a second catheter and the entire volume is delivered within 45 seconds.

In portal vein studies, the dose is also administered via an additional catheter over a 30 min time period.

In the case of oral dosages, the dose is delivered to the back of the mouth with an appropriate size syringe and is followed with 5 ml of tap water to assist swallowing and clearance of the dose from the mouth. Capsules were administered similarly by placing them at the back of the mouth and giving around 5 ml of water to facilitate swallowing.

For all other tests, the dosage is infused into the appropriate intestinal port and flushed with 2-3 ml of sterile saline. The dead space volume of the ports and tubing vary slightly between individuals, but are generally less than 0.5 ml total. Hence, the four to six fold volume flush is accepted as sufficient to insure complete clearing of the dosage from the port and tubing. Systemic blood samples were collected through the portal vein port and catheter.

2) Bioavailability and Absorption of 9-NC

Calculation of 9-NC bioavailability was performed with the AUC_{0-inf} determined with systemic blood samples for both, intravascular and extravascular administration. Systemic blood samples allow the calculation of AUC_{0-inf} after oral capsule administration only and portal vein AUC for all the routes of administration. When AUC_{0-inf} was not possible to calculate (9-NC was not detected), AUC up to the last concentration detected is reported and used in the bioavailability calculation. The average AUC values for 9-NC found after systemic and portal vein samples are shown in Table 22 with their coefficient of variation.

TABLE 22
Mean AUC_{0-inf} values of 9-NC
in systemic and portal vein samples.

| Route of | Systemic AUC | Portal Vein AUC |
|---|---|---|
| Administration | (ng * hr/ml) | (ng * hr/ml) |
| Intravenous - Low | 57.25 ± 5.84 | 45.05 ± 4.08 |
| Intravenous - High | 118.33 ± 21.82 | 93.61 ± 17.02 |
| Oral - capsule | 4.75 ± 4.61 | 11.48 ± 1.50 |
| Oral - suspension | 0.48 ± 0.53 | 11.97 ± 9.88 |
| Intrajejunal | 1.03 ± 1.34 | 4.46 ± 4.00 |
| Intraileum | 1.65 ± 1.15 | 1.76 ± 0.95 |
| Intracolon | 0.07 ± 0.06 | 1.48 ± 0.29 |
| Portal vein | 6.24 ± 2.11 | N/A |

The maximum extravascular bioavailability (4.1%) was achieved after oral administration in capsule form of 9-NC. Direct administration of 9-NC to each intestinal port provided an average value ranging between 3.0 and 0.1%. When 9-NC was given as an infusion in the portal vein, the bioavailability was up to 5.3%. Table 23 shows the results taking W dose as reference (0.1 and 0.2 mg/kg). The bioavailability obtained after capsule administration is higher suggesting that this preparation prevents degradation in the stomach.

TABLE 23
Effect of route and site of administration on bioavailability (%)

| Route of | Bioavailability | Bioavailability | |
|---|---|---|---|
| Administration | (low iv dose) | (high iv dose) | Average |
| Oral - capsule | 4.2 ± 4.0 | 4.1 ± 3.8 | 4.1 |
| Oral - suspension | 0.4 ± 0.6 | 0.4 ± 0.6 | 0.4 |
| Intrajejunal | 3.0 ± 3.2 | 3.0 ± 3.2 | 3.2 |
| Intraileum | 1.6 ± 1.2 | 1.6 ± 1.1 | 1.6 |
| Intracolon | 0.1 ± 0.1 | 0.1 ± 0.1 | 0.1 |
| Portal vein | 5.4 ± 1.4 | 5.2 ± 0.8 | 5.3 |

Metabolism of 9-NC and formation of 9-AC (active metabolite) presented different patterns after intravenous (including PV administration) and any other route of administration tested. The extent of metabolism was evaluated on the bases of 9-AC AUC_{0-inf} values in systemic and portal vein samples (Table 24). In addition, the AUC ratio of metabolite to drug was also calculated with systemic and portal vein samples (Table 25).

TABLE 24
Mean AUC_{0-inf} values of 9-AC
in systemic and portal vein samples.

| Route of Administration | Systemic AUC | Portal Vein AUC |
|---|---|---|
| Intravenous (0.1 mg/kg) | 12.14 ± 5.68 | 10.76 ± 3.02 |
| Intravenous (0.2 mg/kg) | 21.04 ± 2.36 | 25.02 ± 9.87 |
| Oral - capsule | 44.74 ± 24.72 | 66.15 ± 34.53 |
| Oral - suspension | 20.31 ± 6.97 | 34.43 ± 9.52 |
| Intrajejunal | 24.41 ± 7.77 | 50.26 ± 6.65 |
| Intraileum | 21.23 ± 14.94 | 11.83 ± 12.85* |
| Intracolon | 36.91 ± 22.62 | 20.81 ± 9.57 |
| Portal vein | 4.73 ± 0.73 | N/A |

*Calculated from AUC 0-last

TABLE 25
AUC ratio of metabolite to parent drug

| Route of Administration | Systemic | Portal Vein |
|---|---|---|
| Intravenous (0.1 mg/kg) | 0.21 | 0.24 |
| Intravenous (0.2 mg/kg) | 0.18 | 0.27 |
| Oral - capsule | 9.43 | 5.76 |
| Oral - suspension | 42.31 | 2.87 |
| Intrajejunal | 23.70 | 11.26 |
| Intraileum | 12.83 | 6.73 |
| Intracolon | 529.74 | 14.08 |
| Portal vein | 0.76 | N/A |

After IV administration, the AUC of 9-NC and 9-AC are proportional to the dose (at least for the only two doses assayed) and provide a similar ratio with systemic and portal vein sampling. Administration of 9-NC via the portal vein yields a slightly higher ratio. However, when 9-NC was administered orally or via an intestinal port, the ratio metabolite to drug increased dramatically in systemic and portal vein sampling AUC. These changes in AUC ratios after extravascular versus intravenous administration suggest that when 9-NC is given extravascularly and across the intestinal wall the biotransformation becomes more effective. This implies that the metabolism of 9-NC occurs primarily at intestinal level, prior to reaching the systemic circulation. The different ratio in portal vein also suggests that the presentation of 9-NC to the gut wall metabolizing enzymes is different whether the drug is given IV or extravascular.

In portal vein, oral-capsule presents higher 9-AC AUC value that oral suspension (Table 24) but the same 9-NC AUC (Table 22). This suggests that capsule formulation protects 9-NC from stomach degradation that would lead to higher absorption of 9-NC amounts. This could result in more efficient biotransformation and therefore higher 9-AC AUC values.

The high biotransformation of 9-NC to 9-AC in the intestinal wall seems to be a large contributor to its poor bioavailability. However, if the metabolite plays a significant role in the overall pharmacological activity of the drug, then changes in efficacy may be observed depending on the route of administration.

Based on the data displayed in Table 23, for the same formulation (suspension preparation) the highest 9-NC AUC in portal vein is obtained when the drug is given orally, followed by jejunal administration. Within the three intestinal segments, jejunum, ileum and colon, the highest portal vein AUC for 9-NC occurs after jejunum administration. However, the data from oral-suspension seem to indicate that absorption may already begin at the duodenum segment resulting in the higher AUC.

3) Pharmacokinetics of 9-NC After Administration Through Intestinal Ports

The test drug 9-NC was administered in a suspension to the animals via each intestinal port placed in the jejunum, ileum and colon. The dose given was also 0.2 mg/kg. As in the other experiments, blood samples were collected systemic and portal vein and 9-15 NC and 9-AC quantified in plasma by means of LC/MS/MS. The average pharmacokinetic parameters for 9-NC and its metabolite 9-AC are listed in Table 26 (systemic values) and Table 27 (portal vein values).

TABLE 26
Comparison of mean pharmacokinetic parameters found
with systemic samples after administration of 0.2 mg/kg
dose of 9-NC via intestinal ports.

| | JEJUNUM | | ILEUM | | COLON | |
|---|---|---|---|---|---|---|
| Parameter | 9-NC | 9-AC | 9-NC | 9-AC | 9-NC | 9-AC |
| AUC_{0-INF} | 6.66 | 24.41 ± 7.77 | — | 21.23 ± 14.94 | — | 36.91 ± 22.62 |
| (ng * hr/ml) | | | | | | |
| T_{MAX} (hr) | 4.00 ± 3.61 | 8.67 ± 2.31 | 2.25 ± 1.06 | 8.00 ± 0.00 | 0.72 ± 1.11 | 9.33 ± 2.31 |
| C_{MAX} (ng/ml) | 0.96 ± 1.40 | 1.99 ± 1.39 | 0.17 ± 0.01 | 2.04 ± 0.20 | 0.08 ± 0.07 | 1.29 ± 0.63 |
| T_{1/2} (hr) | — | 7.20 ± 5.98 | — | 4.09 ± 4.24 | — | 15.16 ± 14.34 |
| Cl/F (L/hr/kg) | 30 | — | 5.13 | — | — | — |
| MRT (hr) | 54.11 | 15.64 ± 8.69 | 14.11 | 10.64 ± 5.14 | — | 57.62 ± 42.79 |
| Vz (L/kg) | 1469 | — | 1441 | — | — | — |
| AUC_{Last} | 1.03 ± 1.34 | 18.86 ± 5.73 | 1.65 ± 1.15 | 18.69 ± 11.73 | 0.07 ± 0.06 | 19.34 ± 11.92 |
| (ng * hr/ml) | | | | | | |
| F (%) | 3.0 ± 3.2 | — | 1.6 ± 1.2 | — | 0.1 ± 0.1 | — |

TABLE 27
Comparison of mean pharmacokinetic parameters found
with portal vein samples after administration of
0.2 mg/kg dose of 9-NC via intestinal ports.

| | JEJUNUM | | ILEUM | | COLON | |
|---|---|---|---|---|---|---|
| Parameter | 9-NC | 9-AC | 9-NC | 9-AC | 9-NC | 9-AC |
| AUC_{0-INF} | 4.46 ± 4.00 | 50.26 ± 6.65 | 1.76 ± 0.95 | — | 1.48 ± 0.29 | 20.81 ± 9.57 |
| (ng * hr/ml) | | | | | | |
| T_{MAX} (hr) | 0.56 ± 0.42 | 9.33 ± 4.62 | 0.34 ± 0.23 | 6.00 ± 0.00 | 1.67 ± 0.29 | 4.00 ± 2.00 |
| C_{MAX} (ng/ml) | 0.66 ± 0.24 | 3.46 ± 1.22 | 0.65 ± 0.20 | 4.10 ± 2.28 | 0.79 ± 0.61 | 2.28 ± 1.59 |
| T_{1/2} (hr) | 7.59 ± 3.14 | 6.54 ± 1.30 | 6.10 ± 7.50 | — | 1.41 ± 1.75 | 3.98 ± 0.25 |
| Cl/F (L/hr/kg) | — | — | — | — | — | — |
| MRT (hr) | 15.12 ± 1.62 | 13.97 ± 2.62 | 8.65 ± 1.62 | — | 2.21 ± 1.46 | 7.54 ± 0.61 |
| V_{Z} (L/kg) | — | — | — | — | — | — |
| AUC_{Last} | 0.85 ± 0.55 | 28.70 ± 12.16 | 0.87 ± 0.11 | 11.83 ± 12.85 | 0.73 ± 0.63 | 12.69 ± 5.10 |
| (ng * hr/ml) | | | | | | |

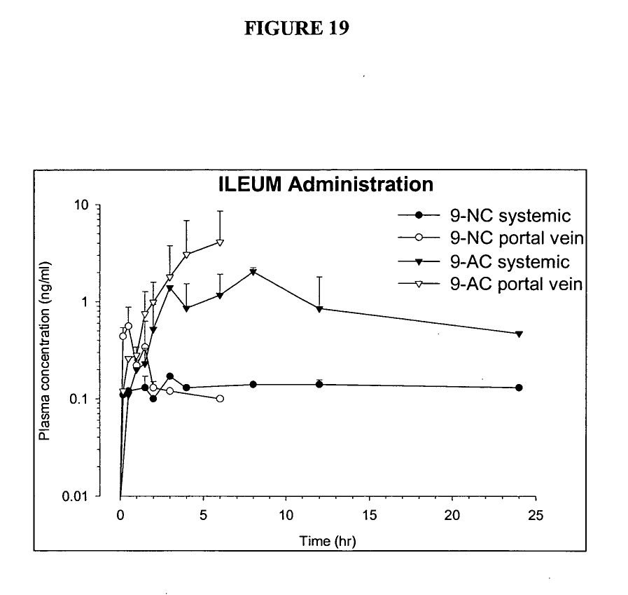

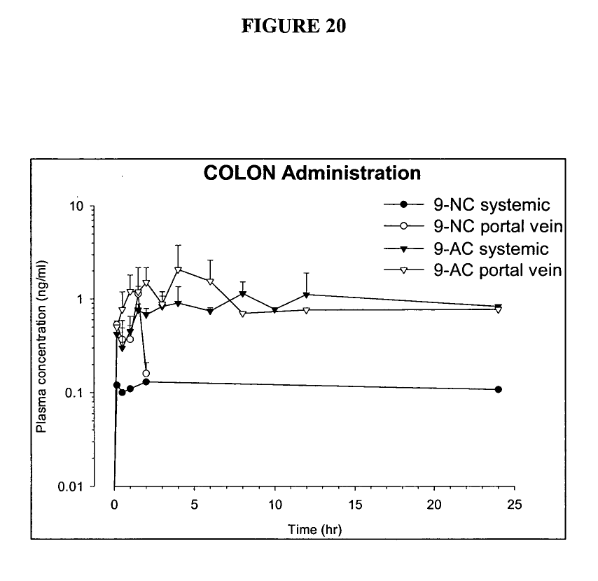

In Table 26 the calculation of bioavailability is included for each of the intestinal segments. Bioavailability was very low in all the intestinal segments and also presented very high variability. In fact, 9-NC was detected with difficulty and its profile was difficult to achieved. In contrast, the results for 9-AC that showed higher values in both systemic and portal vein samples suggesting extensive biotransformation in the gut. The time profile average values obtained after administration via each port is listed below. Table 28 and FIG. 22 display the values after Jejunum administration, Table 29 and FIG. 19 for ileum and Table 30 and FIG. 20 for colon administration.

TABLE 28
Mean 9-NC and 9-AC plasma concentrations (ng/ml)
after 0.2 mg/kg given via the jejunal port.

| | Systemetic | | Portal Vein | |
|---|---|---|---|---|
| Time (hr) | 9-NC | 9-AC | 9-NC | 9-AC |
| Pre-dose | ND | ND | ND | ND |
| 0.17 | BQL | BQL | 0.54 ± 0.22 | 0.10 |
| 0.5 | 0.14 | BQL | 0.41 ± 0.00 | 0.12 |
| 1 | 0.15 | 0.38 | 0.56 ± 0.47 | 0.16 ± 0.10 |
| 2 | 0.14 | 0.32 ± 0.11 | 0.37 ± 0.10 | 0.39 ± 0.04 |
| 3 | 0.15 | 0.47 ± 0.38 | 0.41 | 0.97 ± 0.76 |
| 4 | BQL | 0.89 ± 0.67 | BLOQ | 2.38 ± 2.20 |
| 6 | BQL | 1.59 ± 1.71 | BLOQ | 1.65 ± 1.13 |
| 8 | 1.36 ± 1.73 | 1.71 ± 1.54 | BLOQ | 2.70 ± 0.59 |
| 10 | BQL | 1.81 ± 1.09 | — | — |
| 12 | BQL | 1.03 ± 0.05 | 0.15 | 2.62 ± 0.29 |
| 24 | BQL | 0.45 ± 0.29 | BLOQ | 0.66 |

ND = non detected
BQL = Below quantification limit

TABLE 29
Mean 9-NC and 9-AC plasma concentrations
(ng/ml) after 0.2 mg/kg given via the ileum port.

| | Systemic | | Portal Vein | |
|---|---|---|---|---|
| Time (hr) | 9-NC | 9-AC | 9-NC | 9-AC |
| Pre-dose | ND | ND | ND | ND |
| 0.17 | 0.11 ± 0.01 | BQL | 0.44 ± 0.10 | 0.12 |
| 0.5 | 0.12 | 0.11 | 0.56 ± 0.32 | 0.26 |
| 1 | BQL | 0.20 | 0.22 ± 0.03 | 0.28 ± 0.06 |
| 1.5 | 0.13 ± 0.04 | 0.23 ± 0.10 | 0.34 ± 0.29 | 0.75 ± 0.52 |
| 2 | 0.10 ± 0.00 | 0.52 ± 0.42 | 0.13 ± 0.02 | 0.99 ± 0.60 |
| 3 | 0.17 | 1.40 | 0.12 ± 0.00 | 1.80 ± 1.97 |
| 4 | 0.13 | 0.86 ± 0.67 | BQL | 3.06 ± 3.74 |
| 6 | BQL | 1.17 ± 0.75 | 0.10 | 4.09 ± 4.44 |
| 8 | 0.14 | 2.04 ± 0.20 | — | — |
| 12 | 0.14 ± 0.02 | 0.85 ± 0.95 | — | — |
| 24 | 0.13 | 0.47 | — | — |

ND = non detected
BQL = Below quantification limit

TABLE 30
Mean 9-NC and 9-AC plasma concentrations
(ng/ml) after 0.2 mg/kg given via the COLON port.

| | Systemic | | Portal Vein | |
|---|---|---|---|---|
| Time (hr) | 9-NC | 9-AC | 9-NC | 9-AC |
| Pre-dose | ND | ND | ND | ND |
| 0.17 | 0.117 | 0.42 | 0.53 ± 0.05 | 0.50 ± 0.03 |
| 0.5 | 0.1 | 0.30 ± 0.29 | 0.37 ± 0.12 | 0.77 ± 0.42 |
| 1 | 0.114 | 0.45 ± 0.20 | 0.37 ± 0.15 | 1.20 ± 0.60 |
| 1.5 | BQL | 0.76 ± 0.12 | 1.13 ± 0.23 | 1.19 ± 0.97 |
| 2 | 0.134 | 0.68 ± 0.11 | 0.16 ± 0.05 | 1.50 ± 0.66 |
| 3 | ND | 0.83 ± 0.36 | ND | 0.88 ± 0.19 |
| 4 | BQL | 0.90 ± 0.45 | ND | 2.06 ± 1.69 |
| 6 | BQL | 0.74 ± 0.06 | ND | 1.54 ± 1.06 |
| 8 | BQL | 1.14 ± 0.38 | ND | 0.70 |
| 10 | NM | 0.77 | — | — |
| 12 | BQL | 1.11 ± 0.78 | ND | 0.76 |
| 24 | 0.11 | 0.83 ± 0.00 | ND | 0.77 |

ND = non detected
BQL = Below quantification limit

4. Oral Formulation of Decitabine

The following describes preparation of tablet formulations for decitabine that selectively release drug into the jejunum of the GI tract. These formulations may be used to replace the currently available injectable formulations for chronic administration.

1) Physico-Chemical Properties of Decitabine Affecting Solid Oral Dosage Form Development.

Solid decitabine appears to be white or almost white powder. It is highly soluble in water (˜25 mg/mL) and alcohol, and remains stable at 15-30° C. for 36 months when not exposed to humidity. If exposed to humidity, decitabine forms a monohydrate that corresponds to 7% moisture at equilibrium. Decitabine monohydrate is also stable at room temp.

In comparison the stability of decitabine in aqueous environment is much lower. It starts degrading immediately upon exposure to water. Its degradation is accelerated at acidic and basic pHs. It degrades at pH 7 and 25° C. at the rate of 2.5%/hr, and at pH 7 and 2-8° C. at the rate of about 0.7%/hr.

In a solid form DSC of decitabine indicates a melt at ˜201° C. followed by decomposition. After passing through a screen mill, the final median particle size of decitabine is about 75 μm.

2) Verification of Current Analytical Methods as Suitable for Detecting Decitabine In Its Oral Formulation

Currently an HPLC method is used for drug assay from solution. However, as the tablet formulation contains excipients, a dry powder mixture of decitabine and common excipients such as Microcrystalline cellulose (Avicel PH102, 30% w/w as diluent), Lactose monohydrate (Fastflo 316, 56.5% as diluent), Magnesium stearate (1%, lubricant), Croscarmellose sodium (2%, disintegrant), and colloidal silica (Cab-o-sil, 0.5% as glidant) was made up and evaluated for drug recovery using current method.

Table 31 summarizes results from this study showing that there is no analytical interference of excipients with the drug (i.e., decitabine) and drug recovery was very close to the theoretical value.

Table 31. Excipient interference with drug analysis

TABLE 31
Excipient interference with drug analysis

| | Decitabine | % Recovery of |
|---|---|---|
| Sample ID | Conc.* (mg/ml) | Decitabine |
| Control | 0.22 | 109.13 |
| Formulation | 0.22 | 108.17 |
| Placebo | 0.00 | 1.93 |

*detection at 220 nm wavelength

3) Drug-Excipient Compatibility Testing:

Compatibility of selected excipients used in direct compression tablet formulations with decitabine was evaluated. Since the final formulation is expected to be 10 mg strength, a 10% concentration of drug in blend was used.

TABLE 32
Compositions of drug-excipient blends tested for stability

| | | Sample # | | | | | | | | |
|---|---|---|---|---|---|---|---|---|---|---|
| | | Weight (mg) | | | | | | | | |
| Component | Function | 1 | 2 | 3 | 4 | 5 | 6 | 7 | 8 | 9 |
| Decitabine | Active | 99.3 | 99.8 | 99.5 | 101.4 | 99.9 | 100.5 | 98.7 | 102.6 | 101.0 |
| Avicel PH102 | Diluent | 899.7 | | | | 440.7 | 440.5 | 442.2 | 420.3 | 418.9 |
| Fast Flo lactose 316 | Diluent | | 903.9 | | | 440.0 | | | 419.8 | 421.1 |
| Starch 1500 | Diluent | | | 901.6 | | | 443.6 | | | |
| Calcium phosphate | Diluent | | | | 904.0 | | | 442.2 | | |
| Croscarmellose sodium | Disintegrant | | | | | 19.9 | 21.0 | 21.5 | 20.0 | 21.0 |
| Colloidal silica | Glidant | | | | | | | | 20.8 | 21.1 |
| Magnesium stearate | Lubricant | | | | | | | | 20.7 | |
| Stearic acid | Lubricant | | | | | | | | | 19.2 |

These powder blends were placed at 40° C./75% RH and exposed to moisture by loosening the vial caps, for two weeks. At one and two week time points, samples aliquoted from the vials were analyzed for drug content against the control value (time zero) using HPLC. Chromatographs were analyzed for any degradant peaks as compared to control sample. The degradant peak area is also compared to that from control sample.

TABLE 33
Stability of decitabine in drug-excipient blends

| | T = 0 | | | T = 1 week | | | T = 2 weeks | | | |
|---|---|---|---|---|---|---|---|---|---|---|
| Sample # | % | % | % | % | % | % | % | % | % | |
| 40 C/75% HR | Recovery | Peak | unknown | Recovery | Peak | unknown | Recovery | Peak | unknown | appearance |
| 1 | 88.16 | 99.40 | 0.00 | 88.59 | 98.67 | 0.04 | 85.41 | 99.28 | 0.00 | white powder |
| 2 | 88.90 | 99.27 | 0.06 | 72.11 | 98.36 | 0.04 | 90.82 | 99.24 | 0.00 | white powder |
| 3 | 90.95 | 99.37 | 0.00 | 90.74 | 98.67 | 0.06 | 87.41 | 99.32 | 0.00 | white powder |
| 4 | 94.21 | 99.35 | 0.03 | 98.46 | 98.79 | 0.05 | 94.47 | 98.83 | 0.44 | white powder |
| 5 | 88.29 | 99.24 | 0.09 | 98.82 | 94.43 | 0.00 | 87.25 | 99.3 | 0.00 | white powder |
| 6 | 91.40 | 99.30 | 0.06 | 92.88 | 98.83 | 0.00 | 90.93 | 99.3 | 0.00 | white powder |
| 7 | 99.19 | 89.29 | 0.16 | 89.97 | 98.75 | 0.00 | 95.63 | 99.3 | 0.00 | white powder |
| 8 | 86.93 | 99.24 | 0.08 | 93.78 | 98.80 | 0.00 | 95.18 | 99.3 | 0.00 | white powder |
| 9 | 90.67 | 99.17 | 0.18 | 101.82 | 98.89 | 0.00 | 95.63 | 99.3 | 0.00 | white powder |

As shown in Table 33, results from this study indicate that the potency of drug was maintained, no additional degradants appeared, and visually there was no change in appearance of samples with all the selected excipients.

Accordingly, an embodiment of the tablet formulation of decitabine was designed that contained: decitabine (2% w/w), microcrystalline cellulose (Avicel PH 102, 25%), lactose monohydrate (FastFlo 316, 70.5%), colloidal silica (0.5%), croscarmellose sodium (Ac-Di-Sol, 1%), and magnesium stearate (1%).

4) Placebo Tablets and Coating

Placebo tablets using the above-mentioned formulation, without decitabine, were made by direct compression. Powder blend was manufactured at lab scale by mixing all components except for magnesium stearate for 10 minutes using Turbula shaker-mixer. After the initial blending, the material was passed through 457 micron screen using a Quadro Comil 193AS to disperse material as well as break up any loose aggregates of powder, and lumps of colloidal silica. Magnesium stearate was then added and the material was further blended for 2 minutes using Turbula shaker-mixer.

Stokes 16 punch station, operating with one punch-die (10 mm diameter) was used to make placebo tablets. These tablets were very uniform and close to the target weight of 250 mg, and showed no friability upon standard USP testing. Hardness of uncoated tablets was in the range of 6-9 kp.

5) Enteric Coating of the Tablets

Prior to enteric coating the tablets, a seal coat with hydroxy propylmethylcellulose (HPMC) to a tablet weight gain of 2.9% was applied. This was done to provide additional barrier to moisture during the GI transit of tablet. As decitabine is known to degrade rapidly when solvated in aqueous environment, this seal coat is expected to provide protection to the drug until it reaches the target jejunum area.

TABLE 34
Seal coat formulation

| Component | Function | Quantity | |
|---|---|---|---|
| HPMC | Polymer | 10 | g |
| Tween 80 | Surfactant | 3 | g |
| Triacetin | Plasticizer | 1.5 | ml |
| Alcohol | Solvent | 80 | ml |
| Methylene chloride | Solvent | 70 | ml |

These seal coated tablets were further coated with Eudragit L100 (from Rohm Polymers, specific to jejunum pH of 6.5 as per manufacturer) to varied weight gains of 3, 5, 7.5, and 10%. The composition of enteric coating solution was as follows:

TABLE 35
Enteric coat formulation

| Component | Function | Quantity | |
|---|---|---|---|
| Eudragit L100 | Polymer | 20 | g |
| Triacetin | Plasticizer | 3.2 | g |
| Tween 80 | Surfactant | 3.0 | g |
| Isopropanol | Solvent | 68 | g |
| Acetone | Solvent | 102 | g |

| | | |
|---|---|---|
| Dye | Colorant | q. s. |

During the study, an enteric coat formulation containing talc, anti-tacking agent was found to cause specking on the tablet. Thus, it is preferred that the coating not include talc.

Coating was applied manually using an ‘air paintbrush’ and a rotating pan. With each coating the hardness of tablets increased, tablets with the highest coating of 10% having a hardness of approximately 18 kp.

6) Test of Coated Placebo Tablets

Tablets that were seal coated and coated to various weight gains with enteric coat were tested for disintegration. In this test, a standard equipment meeting USP specifications was employed. Tablets were first placed in pH 1.2 HCl solution for two hours to evaluate if they withstand the acidic environment. The uncoated tablets with or without seal coat disintegrated rapidly with 2-3 minutes. In comparison, enteric-coated tablets were found intact. These tablets then were transferred into pH 6.5 solution and tested for disintegration. Disintegration times depended on the amount of enteric coat—the higher the amount of the coat the longer time it takes to disintegrate. On an average, for 3, 5, 7.5 and 10% weight gains the disintegration times were approximately 13, 20, 22, and 33 minutes, respectively.

It was observed that the even though the tablet contents disintegrated much earlier as seen by seeping of powder through the openings formed at the edges of tablets, the coat remained intact for much longer. Thus it was difficult to find the time of tablet disintegration as defined by USP, which is not to have a coherent core.

7) Preparation of Drug-Excipient Blend and Blend Uniformity Analysis

A potential problem with low dose drug blends (in this case 2% w/w) is non-uniformity of the blend. For the blend uniformity analysis a drug-excipient blend was prepared as follows at lab scale (100 g total).

Decitabine (2.0007 g), colloidal silica (0.501 g), and croscarmellose sodium (1.0 g) were weighed out into a wide mouth glass bottle. These were mixed for 2 minutes on Turbula shaker-mixer. Twenty-five grams of Avicel PH102 was added and mixed for 5 minutes. Lactose (25.5 g) was added and the contents were mixed further for 5 minutes. Additional 45.0 g of lactose was added and mixed for five more minutes. The addition of these components was close to geometric mixing.

The blend then was passed through 457 micron screen using a Quadro Comil 193AS to disperse as well as break up any loose powder aggregates. Magnesium stearate (1.0 g) was added into the blend and mixed on Turbula for 3 minutes.

Three samples collected from distinct areas of blend were analyzed for determining blend uniformity and were found to be uniform.

TABLE 36
Blend Uniformity

| Sample ID | % recovery (mean of two values) |
|---|---|
| A | 103.99 |
| B | 94.41 |
| C | 99.84 |
| Average ± % RSD | 99.41 ± 3.95 |

8) Decitabine Tablet Formulation

Based on the above studies, a tablet formulation of decitabine is designed and prepared that contains a blend of drug substance (2% w/w), microcrystalline cellulose (Avicel PH 102 or similar, 25%), lactose monohydrate (FastFlo 316 or similar, 70.5%), colloidal silica (0.5%), croscannellose sodium (Ac-Di-Sol or similar, 1%), and magnesium stearate (1%).

The tablet is 250 mg with 2% drug load (or 5 mg in each tablet core), 10 mm diameter. It is seal coated with HPMC polymer to approximate weight gain of 3%, and enteric-coated with Eudragit L100 to a weight gain of approximately 3, 5, and 7.5%. Tablets without seal coat are also produced and enteric coated to an approximate weight gain of 3, 5 or 7.5%. The hardness of the uncoated tablet is at least 5 kp and has no or minimal friability. The enteric-coated tablet is preferred not to disintegrate in acidic medium (pH 1.2) for at least 2 hours, but preferred to disintegrate in neutral or weak acidic medium (pH 6.5-7) within 15 minutes.

Uncoated (no seal or enteric coat) tablets, and tablets that have only the seal coat quickly disintegrated (within 1 minute) in acidic disintegration medium of pH 1.2 (0.1 N HCl). Once the tablets (either with seal coat or not) were enteric coated, they did not disintegrate in the same acidic medium for the studied 2 hours. When these tablets were further placed in the pH 6.5 buffer, they disintegrated in the rank order of enteric coat. The higher the enteric coat, higher were the disintegration times. Seal coating the tablet did not affect the disintegration time of the tablets.

In one experiment (Table 37, Formulation # 1), decitabine tablets that were 4 mm in diameter, and approximately 125 mg weight with 2% drug concentration were made as per the blend and tablet manufacturing procedure described above. These tablets were coated with enteric coat to a weight gain of approximately 3%. The tablet hardness was approximately 8 kp. In pH 1.2 medium these tablets were stable for at least 2 hours, and disintegrated in pH 6.5 buffer in an average time of 15 minutes (range of 8-20 minutes,

TABLE 36
Blend Uniformity

| Sample ID | % recovery (mean of two values) |
|---|---|
| A | 103.99 |
| B | 94.41 |
| C | 99.84 |
| Average ± % RSD | 99.41 ± 3.95 |

8. Decitabine Tablet Formulation

Based on the above studies, a tablet formulation of decitabine is designed and prepared that contains a blend of drug substance (2% w/w), microcrystalline cellulose (Avicel PH 102 or similar, 25%), lactose monohydrate (FastFlo 316 or similar, 70.5%), colloidal silica (0.5%), croscarmellose sodium (Ac-Di-Sol or similar, 1%), and magnesium stearate (1%).

The tablet is 250 mg with 2% drug load (or 5 mg in each tablet core), 10 mm diameter. It is seal coated with HPMC polymer to approximate weight gain of 3%, and enteric-coated with Eudragit L100 to a weight gain of approximately 3, 5, and 7.5%. Tablets without seal coat are also produced and enteric coated to an approximate weight gain of 3, 5 or 7.5%. The hardness of the uncoated tablet is at least 5 kp and has no or minimal friability. The enteric-coated tablet is preferred not to disintegrate in acidic medium (pH 1.2) for at least 2 hours, but preferred to disintegrate in neutral or weak acidic medium (pH 6.5-7) within 15 minutes.

Uncoated (no seal or enteric coat) tablets, and tablets that have only the seal coat quickly disintegrated (within 1 minute) in acidic disintegration medium of pH 1.2 (0.1 N HCl). Once the tablets (either with seal coat or not) were enteric coated, they did not disintegrate in the same acidic medium for the studied 2 hours. When these tablets were further placed in the pH 6.5 buffer, they disintegrated in the rank order of enteric coat. The higher the enteric coat, higher were the disintegration times. Seal coating the tablet did not affect the disintegration time of the tablets.

In one experiment (Table 37, Formulation # 1), decitabine tablets that were 4 mm in diameter, and approximately 125 mg weight with 2% drug concentration were made as per the blend and tablet manufacturing procedure described above. These tablets were coated with enteric coat to a weight gain of approximately 3%. The tablet hardness was approximately 8 kp. In pH 1.2 medium these tablets were stable for at least 2 hours, and disintegrated in pH 6.5 buffer in an average time of 15 minutes (range of 8-20 minutes, n=6). Approximately 60% of the theoretical drug was dissolved by 15 minutes in the dissolution test as per USP.

In another experiment (Table 37, Formulation # 2), composition of the decitabine blend was modified to include 10% of Carbopol or HPMC as an exipient to increase gastrointestinal retention. These excipients substituted the lactose component of the composition. Tablets were enteric coated to a weight gain of approximately 2%. The following table shows the composition.

TABLE 37
Decitabine Tablet Composition

| | | | Avicel | | | | | | |
|---|---|---|---|---|---|---|---|---|---|
| | | | PH | | | | | | |
| | | Lactose | 102 | Colloidal | Croscarmellose | | Carbopol | Mg. | |
| | Drug | Monohydrate | % | Silcia | Sodium | HPMC | 934P NF | stearate | Coating |
| # | % w/w | % w/w | w/w | % w/w | % w/w | % w/w | % w/w | % w/w | % w/w |
| 1 | 2 | 60.5 | 25 | 0.5 | 1 | 0 | 10 | 1 | 2 |
| 2 | 2 | 60.5 | 25 | 0.5 | 1 | 10 | 0 | 1 | 2 |

Formulation #1 with Carbopol polymer excipients after coating had shown zero friability and had a mean hardness of 14.5 kp. Tablets from formulation #2 with 10% HPMC also had little friability, and hardness was measured to be a mean value of 8.5 kp. Tablets from these two formulations did not disintegrate for at least one and half hours in acidic disintegration medium of pH 1.2. Placed in pH 6.5 medium, complete disintegration was observed in about 45 minutes. However, tablets started to swell in approximately 4 minutes and the coating was observed to be lost. Tablets stuck to the discs, presumably due to the added polymeric excipients having adhesive properties. In vivo the tablets should adhere to the GI mucous membrane and gain increased GI retention time due to the viscogenic matrix formed due to swelling.

5. Oral Formulation of Pentostatin

The following describes preparation of tablet formulations for pentostatin that selectively release drug into the jejunum of the GI tract. These formulations may be used to replace the currently available injectable formulations for chronic administration.

1) Physico-Chemical Properties of Pentostatin that May Affect Solid Oral Dosage Form Development.

Solid pentostatin appears to be white to off white powder. It freely soluble in water at various pHs, and slightly soluble in ethanol and methanol. The crystalline form of pentostatin is not hygroscopic. DSC scans of pentostatin indicate that onset of melting occurs between 206 and 216° C. Solid pentostatin is stable for at least 12 months up to temperatures of 45° C. At high temperatures (37° C.) and high relative humidity (75% RH), it loses the potency and a visual color change to beige is observed. In aqueous solution, pentostatin at 1 mg/ml was found to be most stable at pH 7 or above and degrade quickly at pHs lower than 4

2) Verification of Non-Interference of Excipients With Drug Analysis

A powder blend of pentostatin and selected excipients of direct compression tablet formulations was analyzed for drug recovery as per the current HPLC assay. Composition of this blend was: decitabine (2 mg or 2%), magnesium stearate (0.2 mg or 1%), colloidal silica (0.1 mg or 0.5%), microcrystalline cellulose (6 mg or 30%), lactose monohydrate (Fastflo 316, 11.3 mg or 56.5%), and croscarmellose sodium (0.4 mg or 2%). Table 24 summarizes the results showing non-interference of excipients with the analysis of drug.

TABLE 38
Excipient interference with drug analysis

| | Pentostatin | % Recovery of |
|---|---|---|
| Sample ID | Conc. (mg/ml)* | pentostatin |
| standard | 1.01600 | 100.00 |
| standard | 0.20348 | 100.14 |
| Standard + excipients | 0.20248 | 99.65 |

*detection at 282 nm wavelength.

3) Drug-Excipient Compatibility Testing

Compatibility of the selected excipients used in direct compression tablet formulations with pentostatin was evaluated, essentially the same way as done with decitabine.

TABLE 39
Compositions of pentostatin-excipient blends tested for stability

| | | Sample # | | | | | | | | |
|---|---|---|---|---|---|---|---|---|---|---|
| | | Weight (mg) | | | | | | | | |
| Component | Function | 1 | 2 | 3 | 4 | 5 | 6 | 7 | 8 | 9 |
| Pentostatin | Active | 100.4 | 101.6 | 100.5 | 98.5 | 100.2 | 100.5 | 99.8 | 100.8 | 100.2 |
| Avicel PH102 | Diluent | 899.0 | | | | 442.4 | 440.5 | 441.8 | 421.1 | 419.2 |
| Fast Flo lactose 316 | Diluent | | 903.7 | | | 442.2 | | | 421.2 | 421.4 |
| Starch 1500 | Diluent | | | 905.3 | | | 439.5 | | | |
| Calcium phosphate | Diluent | | | | 898.8 | | | 442.1 | | |
| Croscarmellose sodium | Disintegrant | | | | | 21.2 | 20.2 | 21.3 | 21.2 | 20.7 |
| Colloidal silica | Glidant | | | | | | | | 20.6 | 22.5 |
| Magnesium stearate | Lubricant | | | | | | | | 22.0 | |
| Stearic acid | Lubricant | | | | | | | | | 21.6 |

These powder blends were placed at 40° C./75% RH and exposed to moisture by loosening the vial caps for two weeks. At one and two week time points, samples aliquoted from the vials were analyzed for drug content against the control value (time zero) using HPLC. Chromatographs were analyzed for any degradant peaks as compared to control sample. The degradant peak area is also compared to that from control sample.

TABLE 40
Drug-excipient stability studies

| | | Time = 0 initial | | Time = 1 week | | Time = 2 weeks | |
|---|---|---|---|---|---|---|---|
| Formulation | Condition | Potency | TRS | Potency | TRS | Potency | TRS |
| Pentostatin + Avicel | | | | | | | |
| Sample 1-a | 25° C. | 90.81% | 2.47% | | | | |
| Sample 1-b | 25° C. | 95.53% | 2.30% | | | | |
| Sample 1-a | 40° C. | | | 100.39% | 2.47% | 90.30% | 2.52% |
| Sample 1-b | 40° C. | | | 98.13% | 2.50% | 90.75% | 2.54% |
| Pentostatin + Lactose | | | | | | | |
| Sample 2-a | 25° C. | 96.42% | 2.39% | | | | |
| Sample 2-b | 25° C. | 93.19% | 2.48% | | | | |
| Sample 2-a | 40° C. | | | 90.67% | 2.50% | 91.61% | 2.49% |
| Sample 2-b | 40° C. | | | 94.38% | 2.43% | 91.78% | 2.49% |
| | | | | slight color | | light beige | |
| | | | | change | | color | |
| Pentostatin + Starch | | | | | | | |
| Sample 3-a | 25° C. | 95.62% | 2.29% | | | | |
| Sample 3-b | 25° C. | 85.63% | 2.43% | | | | |
| Sample 3-a | 40° C. | | | 96.69% | 2.29% | 97.73% | 2.33% |
| Sample 3-b | 40° C. | | | 97.19% | 2.37% | 92.61% | 2.36% |
| Pentostatin + | | | | | | | |
| Calcium phosphate | | | | | | | |
| Sample 4-a | 25° C. | 79.62% | 2.36% | | | | |
| Sample 4-b | 25° C. | 73.69% | 2.48% | | | | |
| Sample 4-a | 40° C. | | | 86.93% | 2.42% | 97.19% | 2.28% |
| Sample 4-b | 40° C. | | | 97.33% | 2.36% | 85.50% | 2.36% |
| Pentostatin + Avicel, | | | | | | | |
| Lactose, Croscarmellose | | | | | | | |
| Sample 5-a | 25° C. | 85.26% | 2.40% | | | | |
| Sample 5-b | 25° C. | 86.56% | 2.37% | | | | |
| Sample 5-a | 40° C. | | | 91.57% | 2.64% | 92.07% | 2.57% |
| Sample 5-b | 40° C. | | | 92.67% | 2.65% | 95.30% | 2.62% |
| | | | | slight color | | light beige | |
| | | | | change | | color | |
| Pentostatin + Avicel, | | | | | | | |
| Starch, Croscarmellose | | | | | | | |
| Sample 6-a | 25° C. | 96.33% | 2.32% | | | | |
| Sample 6-b | 25° C. | 92.04% | 2.35% | | | | |
| Sample 6-a | 40° C. | | | 91.56% | 2.38% | 95.88% | 2.41% |
| Sample 6-b | 40° C. | | | 97.56% | 2.36% | 92.44% | 2.45% |
| Pentostatin + Avicel, | | | | | | | |
| Calcium Phosphate, | | | | | | | |
| Croscarmellose | | | | | | | |
| Sample 7-a | 25° C. | 90.83% | 2.39% | | | | |
| Sample 7-b | 25° C. | 89.25% | 2.35% | | | | |
| Sample 7-a | 40° C. | | | 99.35% | 2.37% | 96.42% | 2.47% |
| Sample 7-b | 40° C. | | | 91.47% | 2.39% | 92.31% | 2.58% |
| | | | | | | slight color | |
| | | | | | | change | |
| Pentostatin + Avicel, | | | | | | | |
| Lactose, Croscarmellose, | | | | | | | |
| Colloidal Silica, Mg | | | | | | | |
| Stearate | | | | | | | |
| Sample 8-a | 25° C. | 95.08% | 2.24% | | | | |
| Sample 8-b | 25° C. | 87.03% | 2.37% | | | | |
| Sample 8-a | 40° C. | | | 99.67% | 2.41% | 89.36% | 2.51% |
| Sample 8-b | 40° C. | | | 101.32% | 2.42% | 94.47% | 2.48% |
| | | | | slight color | | light beige | |
| | | | | change | | color | |
| Pentostatin + Avicel, | | | | | | | |
| Lactose, Croscarmellose, | | | | | | | |
| Colloidal Silica, Stearic | | | | | | | |
| Acid | | | | | | | |
| Sample 9-a | 25° C. | 94.43% | 2.36% | | | | |
| Sample 9-b | 25° C. | 93.80% | 2.31% | | | | |
| Sample 9-a | 40° C. | | | 100.26% | 2.37% | 84.78% | 2.73% |
| Sample 9-b | 40° C. | | | 101.33% | 2.41% | 96.55% | 2.50% |

Even though the variation in drug potency values and total related substances was not significant, it was observed that with many samples the color changed. In continuation of these stability studies, two more disintegrants (polyplasdone and sodium starch glycolate) were added to drug with or without diluents (Avicel and pregelatinized starch). These results are shown Table 41.

TABLE 41
Drug-excipient stability studies

| | | Time = 0 initial | | Time = 2 weeks | |
|---|---|---|---|---|---|
| Formulation | Condition | Potency | TRS | Potency | TRS |
| Pentostatin + | | | | | |
| Polyplasdone | | | | | |
| Sample 1 | 25° C. | 93.65% | 2.50% | | |
| Sample 1 | 40° C. | | | 85.46% | 2.87% |
| | | | | no color change | |
| Pentostatin + Na | | | | | |
| Starch Glycolate | | | | | |
| Sample 2 | 25° C. | 105.33% | 2.53% | | |
| Sample 2 | 40° C. | | | 87.34% | 2.00% |
| | | | | slight color change | |
| Pentostatin + | | | | | |
| Polyplasdone, | | | | | |
| Avicel, Starch | | | | | |
| Sample 3 | 25° C. | 100.74% | 2.31% | | |
| Sample 3 | 40° C. | | | 89.28% | 2.64% |
| | | | | no color change | |
| Pentostatin + Na | | | | | |
| Starch Glycolate, | | | | | |
| Avicel, Starch | | | | | |
| Sample 4 | 25° C. | 103.45% | 2.35% | | |
| Sample 4 | 40° C. | | | 87.64% | 2.58% |

no color change

As shown in Table 41, there was no color change in samples 1, 3 and 4. As no significant reduction in potency or generation of extra or new degradants was observed, color change was not considered to be a significant factor in formulation development. In addition, drug when exposed to high temperature and relative humidity is also known to change color.

4) Blend Development and Manufacture

As described above animal studies have shown a significant increase in oral bioavailability from jejunum area when the drug was given as a pH 7 buffered solution as compared to in normal saline. Accordingly, the tablet formulation of pentostatin is designed to include powder buffer salts to make the tablet blend have a pH of 7 when dissolved in the intestinal Oejunum) fluids. Assuming that in the immediate environment of tablet in the jejunum is about 3-5 ml of liquid, the amounts of buffer and other excipients were calculated, and are given below.

Avicel PH 102—62% w/w (diluent)

Starch 1500—20% (diluent and disintegrant)

KH₂PO₄—4% (buffer)

Na₂HPO₄—12.5% (buffer)

Colloidal silica—0.5% (glidant)

Stearic acid—1% (lubricant)

No specific disintegrant was added since starch 1500 is known to impart disintegrant properties to the blend.

Blend preparation was same as that used in case of decitabine blend described above.

5) Placebo Tablets

Placebo tablets using the above-described blend were made using Stokes 16 station press, and a single punch and die. Tablets were determined to be close to the target weight of 250 mg, with a low relative standard deviation. These tablets were hard and showed no friability.

6) Tablet Formulation of Pentostatin

Based on the above studies, an embodiment of oral formulation of pentostatin is designed and prepared. The tablet blend includes: Pentostatin (2% w/w), Avicel PH 102—62% w/w (diluent), Starch 1500—20% (diluent and disintegrant), KH₂PO₄—4% (buffer), Na₂HPO₄—12.5% (buffer), Colloidal silica—0.5% (glidant), and Stearic acid—1% (lubricant).

The tablet is 250 mg with 2% pentostatin (or 5 mg in each tablet core), 10-13 mm diameter. It can be seal coated with HPMC polymer to approximate weight gain of 3%, and enteric coated with Eudragit L1OO to a weight gain of 5%. The hardness of the uncoated tablet is at least 8 kp and has no or minimal friability. The enteric coated tablet is preferred not to disintegrate in acidic medium (pH 1.2) for at least 2 hours, but preferred to disintegrate in neutral or weak acidic medium (pH 6.5-7) within 15 minutes.

6. Oral Formulation of 9-Nitro-Camptothecin (9-NC)

Solid 9-NC appears to be fine, yellow, crystalline, powder. It is practically insoluble in water and alcohol. The solid pure crystal form of 9-NC is stable at 15-30° C. for over 24 months; and 9NC in powder form is stable at 80° C. for at least two weeks. In a pure crystal form, 9-NC is not hygroscopic, even in a 95% RH environment. Other forms of 9-NC are either hydrates or solvates. The polarity of 9NC is indicated by having an octanol water coefficient of about 17.6

DSC of the solid pure crystal form of 9NC indicates a melt onset at ˜250° C. followed by decomposition at about 270° C. Polymorph screen indicates that the solid pure crystal form of 9NC is the most stable.

As for the stability of 9NC in aqueous environment, it has been found that in an aqueous environment at pH 7 ring E of 9-NC opens to yield the open carboxylate form. However, due to its low aqueous solubility, the percent conversion is small. 9-NC starts degrading immediately at basic pHs above 9, and is stable at acidic pHs.

The 9-NC drug substance is not milled and the median particle size of the unmilled drug is between 75-200 μm. For oral dosage form development, the drug can be micronized using high pressure jet milling. Micronization of 9-NC to a median particle size of 2-11 μm has shown that the polymorph is not changed post micronization.

An embodiment of oral formulation of 9-nitro-camptothecin (9NC) is designed and prepared. The tablet blend includes: 9NC (1-10% w/w), Avicel PH 102—62% w/w as diluent (alternatively or additionally, lactose monohydrate, pre-gelatinized starch, or calcium phosphate), Starch 1500—20% as disintegrant (alternatively or additionally, croscarmellose sodium, polyplasdone, or sodium starch glycolate), Colloidal silica—0.5% as glidant, and Stearic acid—1% as lubricant (alternatively or additionally, magnesium stearate).

Since 9NC is not hygroscopic and does not degrade in acidic environment, the tablet may not need be seal coated (e.g., with HPMC polymer), but can be enteric coated with Eudragit L100 to a weight gain of 5%. The enteric coated tablet is preferred not to disintegrate in acidic medium (pH 1.2) for at least 2 hours, but preferred to disintegrate in neutral or weak acidic medium (pH 6.5-7) within 15 minutes.

It will be apparent to those skilled in the art that various modifications and variations can be made in the compounds, compositions, kits, and methods of the present invention without departing from the spirit or scope of the invention. Thus, it is intended that the present invention cover the modifications and variations of this invention provided they come within the scope of the appended claims and their equivalents.

What is claimed is:

1. A pharmaceutical composition, comprising:
a water-soluble, acid-labile drug enteric-coated with a enteric coating material that dissolves at pH above about 5.2.

2. The pharmaceutical composition of claim 1, wherein the solubility of the drug is above 1 mg/ml in water or aqueous solution.

3. The pharmaceutical composition of claim 1, wherein the solubility of the drug is above 10 mg/ml in water or aqueous solution.

4. The pharmaceutical composition of claim 1, wherein the drug is labile at pH lower than 5.0.

5. The pharmaceutical composition of claim 1, wherein the drug is labile at pH lower than 2.0.

6. The pharmaceutical composition of claim 1, wherein the drug is a cytidine analog.

7. The pharmaceutical composition of claim 6, wherein the cytidine analog is 5-azacytidine or decitabine.

8. The pharmaceutical composition of claim 1, wherein the drug is a 2′-deoxyadenosine analog.

9. The pharmaceutical composition of claim 8, wherein the 2′-deoxyadenosine analog is pentostatin, fludarabine, or 2-chloro-2′-deoxyadenosine.

10. The pharmaceutical composition of claim 1, wherein the enteric coating material is pH-sensitive and dissolves at pH above about 5.5.

11. The pharmaceutical composition of claim 1, wherein the enteric coating material is pH-sensitive and dissolves at pH above about 6.4.

12. The pharmaceutical composition of claim 1, wherein the enteric coating material is pH-sensitive and dissolves in normal human jejunum juice.

13. The pharmaceutical composition of claim 1, wherein the enteric coating material is pH-sensitive and the pharmaceutical composition substantially disintegrates in an aqueous medium at or above pH 5.5 within 1 hour.

14. The pharmaceutical composition of claim 1, wherein the enteric coating material is pH-sensitive and the pharmaceutical composition substantially disintegrates in an aqueous medium at or above pH 5.5 within 30 minutes.

15. The pharmaceutical composition of claim 1, wherein the coating material comprises an agent selected from the group consisting of cellulose phthalates, EUDRAGIT polymers, polyvinylacetate phthalate, SHELLAC, and cellulose acetate phthalate.

16. The pharmaceutical composition of claim 1, wherein the enteric coating material comprises EUDRAGIT L100.

17. The pharmaceutical composition of claim 1, wherein the enteric coating material comprises EUDRAGIT L100-55.

18. The pharmaceutical composition of claim 1, wherein the enteric coating material further comprises triacetin and TWEEN 80.

19. The pharmaceutical composition of claim 1, wherein the pharmaceutical composition does not substantially disintegrate in an acidic, aqueous medium at pH 1.0-3.0 for at least 1 hour.

20. The pharmaceutical composition of claim 1, wherein the pharmaceutical composition does not substantially disintegrate in an acidic, aqueous medium at pH 1.2-1.5 for at least 2 hours.

21. The pharmaceutical composition of claim 1, wherein the pharmaceutical composition disintegrates substantially in an aqueous medium at pH 5.2-7.5 within 30 minutes.

22. The pharmaceutical composition of claim 1, wherein the amount of the enteric-coating material is 1-8% w/w in the composition.

23. The pharmaceutical composition of claim 1, wherein the pharmaceutical composition is in a form of tablet.

24. The pharmaceutical composition of claim 23, wherein the hardness of the tablet without the enteric-coat is at least 5 kp.

25. The pharmaceutical composition of claim 1, wherein the concentration of the drug is 0.1-10% w/w in the composition.

26. The pharmaceutical composition of claim 1, wherein the drug is contained in a drug core that is enteric-coated with the coating material.

27. The pharmaceutical composition of claim 26, further comprising:
a seal-coating material that coats the surface of the drug core and seals the drug from the moisture.

28. The pharmaceutical composition of claim 27, where the seal-coating material comprises hydroxy propylmethylcellulose.

29. The pharmaceutical composition of claim 27, where the seal-coating material comprises hydroxy propylmethylcellulose, TWEEN 80 and triacetin.

30. The pharmaceutical composition of claim 1, further comprising:
buffer salt in an amount sufficient to maintain the pH of the local environment to be 5.2-7.0 when the pharmaceutical composition is dissolved in the GI tract.

31. The pharmaceutical composition of claim 30, wherein the buffer salt is sodium or potassium phosphate.

32. The pharmaceutical composition of claim 1, further comprising:
one or more pharmaceutically acceptable excipient selected from the group consisting of diluent, lubricant, disintegrant, glidant or retention-enhancing excipient.

33. The pharmaceutical composition of claim 32, wherein the one or more excipient is blended with drug and the mixture of which forms a drug core.

34. The pharmaceutical composition of claim 33, wherein the drug core is directly coated with the enteric coat.

35. The pharmaceutical composition of claim 33, wherein the drug core is first sealed with a seal-coating material and then coated with the enteric coat.

36. The pharmaceutical composition of claim 32, wherein the diluent is selected from the group consisting of microcrystalline cellulose, lactose monohydrate, starch, gelatin, gum, tragacanth, calcium phosphate, sucrose, mannitol, sorbitol, and dextrose.

37. The pharmaceutical composition of claim 32, wherein the lubricant is selected from the group consisting of magnesium stearate, stearic acid, and calcium stearate.

38. The pharmaceutical composition of claim 32, wherein the disintegrant is selected from the group consisting of croscarmellose sodium, polyvinylpyrrolidone, polyvinylpolypyrrolidone, agar, alginic acid, a salt of alginic acid, sodium alginate, sodium starch glycolate, and starch.

39. The pharmaceutical composition of claim 32, wherein the disintegrant is selected from the group consisting of colloidal silica, talc, cornstarch, and syloid.

40. The pharmaceutical composition of claim 32, wherein the retention-enhancing excipient is selected from the group consisting of bioadhesive polymers, mucoadhesive polymers, swelling hydrogels, and viscogenic agents.

41. The pharmaceutical composition of claim 32, wherein the retention-enhancing excipient is selected from the group consisting of carboxyvinyl polymer, methyl cellulose, hydroxypropyl methylcellulose, and polycarbophil.

42. The pharmaceutical composition of claim 32, wherein the one or more excipient is a combination of microcrystalline cellulose, starch, colloidal silica and stearic acid.

43. The pharmaceutical composition of claim 42, wherein the drug and the one or more excipient are blended together to form a drug core which is then enteric-coated with the enteric coating material.

44. A method for treating a patient having a disease associated with abnormal cell proliferation, comprising:
orally administering to the patient a pharmaceutical composition comprising a water-soluble, acid-labile drug enteric-coated with an enteric coating material that dissolves at pH above about 5.2.

45. The method of claim 44, wherein the disease associated with abnormal cell proliferation is selected from the group consisting of hematological disorders, benign tumors, cancer, restenosis, and inflammatory diseases.

46. A method for treating a patient having an autoimmune disease, comprising:
orally administering to the patient a pharmaceutical composition comprising a water-soluble, acid-labile 2′-deoxyadenosine analog enteric-coated with a coating material that dissolves at pH above about 5.2.

47. The method of claim 46, wherein the autoimmune disease is selected from the group consisting of Sjogren's disease, systemic lupus erythematodes, glomerulonephritis, rheumatoid arthritis, generalized necrotizing angitis, granulomatous angitis, autoimmune thyroiditis, diabetes mellitus, myasthenia gravis, and multiple sclerosis.

48. The method of claim 46, wherein the 2′-deoxyadenosine analog is pentostatin.

49. A pharmaceutical composition, comprising: a camptothecin compound enteric-coated with an enteric coating material that dissolves at pH above about 5.2.

50. The pharmaceutical composition of claim 49, wherein the enteric coating material is pH-sensitive and dissolves at pH above about 5.8.

51. The pharmaceutical composition of claim 49, wherein the enteric coating material is pH-sensitive and dissolves at pH above about 6.4.

52. The pharmaceutical composition of claim 49, wherein the enteric coating material is pH-sensitive and dissolves in normal human jejeunum juice.

53. The pharmaceutical composition of claim 49, wherein the enteric coating material is pH-sensitive and dissolves at pH above about 5.8.

54. The pharmaceutical composition of claim 49, wherein the enteric coating material is selected from the group consisting of cellulose phthalates, EUDRAGIT polymers, polyvinylacetate phthalate, SHELLAC, and cellulose acetate phthalate.

55. The pharmaceutical composition of claim 49, wherein the enteric coating material is EUDRAGIT L100.

56. The pharmaceutical composition of claim 49, wherein the enteric coating material is EUDRAGIT L100-55.

57. The pharmaceutical composition of claim 49, wherein the enteric coating material further comprises triacetin and TWEEN 80.

58. The pharmaceutical composition of claim 49, wherein the camptothecin compound is selected from the group consisting of 9-nitro-20(S)-camptothecin, 9-amino-20(S)-camptothecin, 9-methyl-camptothecin, 9-chloro-camptothecin, 9-flouro-camptothecin, 7-ethyl camptothecin, 10-methyl-camptothecin, 10-chloro-camptothecin, 10-bromo-camptothecin, 10-fluoro-camptothecin, 9-methoxy-camptothecin, 11-fluoro-camptothecin, 7-ethyl-10-hydroxy camptothecin, 10,11-methylenedioxy camptothecin, and 10,11-ethylenedioxy camptothecin, and 7-(4-methylpiperazinomethylene)-10,11-methylenedioxy camptothecin.

59. The pharmaceutical composition of claim 49, wherein the camptothecin compound is water-insoluble.

60. The pharmaceutical composition of claim 49, wherein the camptothecin compound is 9-nitro-20(S)-camptothecin.

61. The pharmaceutical composition of claim 49, further comprising one or more pharmaceutically acceptable excipient selected from the group consisting of diluent, lubricant, disintegrant, glidant or retention-enhancing excipient.

62. The pharmaceutical composition of claim 61, wherein the one or more excipient is blended with the drug and the mixture of which forms a drug core.

63. The pharmaceutical composition of claim 62, wherein the camptothecin compound is water-insoluble and the drug core is directly enteric coated with the enteric coating material.

64. The pharmaceutical composition of claim 62, wherein the camptothecin compound is water-soluble, and the drug core is first sealed with a seal-coating material and then coated with the enteric coat.

65. The pharmaceutical composition of claim 61, wherein the diluent is selected from a group consisiting of microcrystalline cellulose, lactose monohydrate, starch, gelatin, gum, tragacanth, calcium phosphate, sucrose, mannitol, sorbitol, and dextrose.

66. The pharmaceutical composition of claim 61, wherein the lubricant is selected from the group consisting of magnesium stearate, stearic acid, and calcium stearate.

67. The pharmaceutical composition of claim 61, wherein the disintegrant is selected from the group consisting of croscarmellose sodium, polyvinylpyrrolidone, polyvinylpolypyrrolidone, agar, alginic acid, a salt of alginic acid, sodium alginate, sodium starch glycolate, and starch.

68. The pharmaceutical composition of claim 50, wherein the disintegrant is selected from the group consisting of colloidal silica, talc, cornstarch, and syloid.

69. The pharmaceutical composition of claim 50, wherein the retention-enhancing excipient is selected from the group consisting of bioadhesive polymers, mucoadhesive polymers, swelling hydrogels, and viscogenic agents.

70. The pharmaceutical composition of claim 51, wherein the retention-enhancing excipient is selected from the group consisting of carboxyvinyl polymer, methyl cellulose, hydroxypropyl methylcellulose, and polycarbophil.

71. A method for treating a patient having a disease associated with abnormal cell proliferation, comprising:
orally administering to the patient a pharmaceutical composition comprising a camptothecin compound enteric-coated with a enteric coating material that dissolves at pH above about 5.2.

72. The method of claim 50, wherein the disease associated with abnormal cell proliferation is selected from the group consisting of hematological disorders, benign tumors, cancer, restenosis, and inflammatory diseases.
